(19) United States
(12) Patent Application Publication
(10) Pub. No.: US 2023/0270890 A1
(43) Pub. Date: Aug. 31, 2023

(54) FLUORESCENT COMPOUND FOR DETECTING BIOLOGICAL MATERIAL AND PREPARATION METHOD THEREOF

(71) Applicant: BIOACTS CORPORATION, Incheon (KR)

(72) Inventors: JIN WOO PARK, Incheon (KR); SU JUNG JANG, Incheon (KR); EUNAE JEONG, Incheon (KR); JIHYUN LEE, Incheon (KR); KIWON KIM, Incheon (KR); EUNSEO YU, Incheon (KR); NURI CHOI, Incheon (KR); GYEONG RIM SHIN, Incheon (KR)

(21) Appl. No.: 18/073,624

(22) Filed: Dec. 2, 2022

(30) Foreign Application Priority Data
Feb. 27, 2022 (KR) 10-2022-0025605

(51) Int. Cl. A61K 49/00

(52) U.S. Cl. CPC A61K 49/0032

(57) ABSTRACT

Provided is a fluorescent compound for labeling a biomaterial having the following Chemical Formula 1:

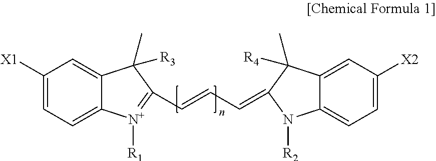

In Chemical Formula 1 above,
X1 and X2 are the same as or different from each other, and each independently selected from H, —SO₃⁻ and SO₃H,
R₁ and R₂ are the same as each other or each independently selected from C₁₋₇ alkyl, C₈₋₁₈ alkyl, —(CH₂)ₘSO₃⁻, —(CH₂)ₘSO₃H, and

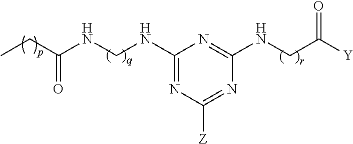

R₃ and R₄ are the same as or different from each other and each independently selected from C₁₋₇ alkyl, —(CH₂)ₘCOOZ and

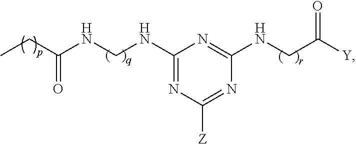

R₃ and R₄ are simultaneously not any one selected from —(CH₂)ₘCOOZ and

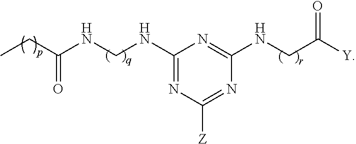

### CROSS-REFERENCE TO RELATED APPLICATIONS

This application claims priority from and the benefit of Korean Patent Application No. 10-2022-0025605, filed on Feb. 27, 2022, which is hereby incorporated by reference for all purposes as if fully set forth herein.

### BACKGROUND

### Field

The present disclosure relates to a fluorescent compound, which is a compound that is useful for a fluorescent diagnosis composition capable of prediction, diagnosis, treatment and prognosis of diseases.

The fluorescent compound provided in exemplary embodiments of the present invention relates to a fluorescent compound which improves the low fluorescence efficiency of conventional cyanine compounds and comprises a triazine structure having an amino-sulfonic acid group that replaces a cyanine-based substituent as a linker.

The fluorescent compound provided in exemplary embodiments of the present invention has less noise and high fluorescent efficiency to improve the efficiency of fluorescent signals when a desired biomaterial is detected using a fluorescence diagnostic composition of the present invention and thus can more accurately diagnose the biomaterial than the related art.

### Description of the Related Art

Since biomaterial itself has weak fluorescence or no fluorescence in visible and near-infrared regions, in the bio field, in order to observe biological phenomena at cellular and subcellular levels in vivo or in vitro or to make images and obtain optical images of a diseased area by being projected into a living body, imaging data have been obtained through a variety of methods using a fluorescent dye or a specific biomaterial pre-labeled with the fluorescent dye in the biomaterial with optical equipment.

Various optical analysis devices used in the bio field select a fluorescent dye with an excitation wavelength and an emission wavelength suitable for observing fluorescence according to embedded light sources and filters as a basic material or reagent.

In general, most of fluorescent dyes used for labeling biomolecules such as proteins or peptides include structures, such as anthranilate, 1-alkylthic isoindoles, pyrrolinones, bimanes, benzoxazole, benzimidazole, benzofurazan, naphthalenes, coumarins, cyanine, stilbenes, carbazoles, phenanthridine, anthracenes, bodipy, fluoresceins, eosins, rhodamines, pyrenes, chrysenes and acridines.

When selecting a fluorescent dye structure usable in the bio field from a plurality of fluorescent chromophores illustrated above, generally, it is important to emit strong fluorescence when most of biomolecules exist in a medium, that is, an aqueous solution and an aqueous buffer, and to have excitation and fluorescence wavelengths suitable for fluorescence equipment.

Dyes that may be mainly applied in the bio technology field need to preferably have less photobleaching and quenching in aqueous or hydrophilic conditions, to have a large molecular extinction coefficient so as to absorb a large amount of light, to be in the visible or near-infrared region of 500 nm or more far from the fluorescence range of the biomolecule itself, and to be stable under various pH conditions. However, structures of dyes usable for labeling biomolecules capable of satisfying the limitations are limited.

Fluorescent chromogens that meet these requirements include cyanine, rhodamine, flocseine, bodipy, coumarin, acridine, and pyren derivatives, and introduce functional groups so as to bind to a dye alone or a specific substituent in a biomolecular structure. Among them, xanthane-based flocseine and rhodamine, and polymethine-based cyanine derivative dye compounds are mainly commercialized.

In particular, the dye compound having the cyanine chromophore has an advantage that it is easy to synthesize compounds various absorption/excitation wavelengths. In addition, generally, since the dye compound having the cyanine chromophore is excellent in optical and pH stability, has narrow absorption and emission wavelength ranges, and has a fluorescent area of 500 to 800 nm, the dye compound is not overlapped with the self-fluorescent region of the biomolecule to be easily analyzed and has a slight difference according to a solvent and solubility characteristics, but has many advantages such as representing high molar adsorption coefficient, and thus is frequently used for biological applications.

In addition, the dye compound having the cyanine chromophore can also be usefully employed for optical filters for image display devices or resin compositions for laser fusion. The compound having a large intensity of absorption in specific light has been widely used as an optical element of an optical filter for an image display device such as a liquid crystal display device, a plasma display panel, an electroluminescence display, a cathode tube display panel, and a fluorescent display tube or an optical recording medium of DVD±R and the like. The optical filter has required a function of selectively absorbing light having unnecessary wavelengths, and simultaneously has required light absorption of wavelengths of 480 to 500 nm and 540 to 560 nm to prevent reflections or glare of external light such as fluorescent light, and has required a function of selectively absorbing wavelengths of infrared light in order to increase the image quality.

Therefore, in order to usefully apply the dyes industrially, it has been continuously required to develop novel dyes that have excellent optical and pH stability, have a narrow absorption/emission wavelength range in a specific wavelength range, and exhibit a high molar absorption coefficient.

The above-stated technical description is the background art for helping in the understanding of the present disclosure, and does not mean a conventional technology widely known in the art to which the present disclosure pertains.

### SUMMARY

An object of the present invention is to provide a fluorescent compound, a preparation method of the compound or a fluorescent diagnostic composition including the compound capable of being used as a contrast medium composition by improving further the fluorescent intensity in a florescent region of 500 to 800 nm while having excellent optical and pH stability and a narrow absorption/emission wavelength range, and particularly, improving the fluorescence by introducing a linker having a triazine structure substituted with a hydroxyl group to a cyanine-based fluorescent compound.

In order to solve the above-described problems, the inventors of the present application have developed a fluorescent compound represented by the following Chemical Formula 1.

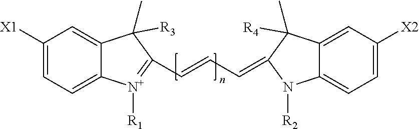

In Chemical Formula 1 above,

X1 and X2 are the same as or different from each other, and each independently selected from H, —SO₃⁻ and SO₃H,

R₁ and R₂ are the same as each other or each independently selected from C₁₋₇ alkyl, C₈₋₁₈ alkyl, —(CH₂)ₘSO₃⁻, —(CH₂)ₘSO₃H, and

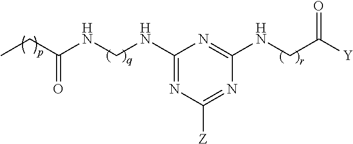

R₃ and R₄ are the same as or different from each other and each independently selected from C₁₋₇ alkyl, —(CH₂)ₘCOOZ and

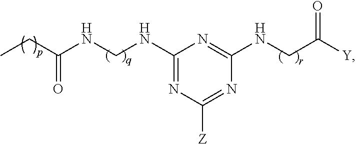

R₃ and R₄ are not any one simultaneously selected from —(CH₂)ₘCOOZ and

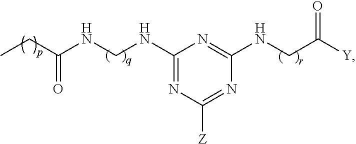

wherein,

n is an integer of 0 to 6,

m is an integer of 1 to 7,

p is an integer of 1 to 10,

q is an integer of 0 to 10,

r is an integer of 1 to 10, and

Z is OH or NH(CH₂)ₛSO₃H,

s is an integer of 1 to 7,

Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀₋₁₀ alkylamyl group, a vinylsulfonyl group, a vinylsulfonyl C₀₋₆ alkylaminyl group and an amino C₀₋₆ alkyl.

According to exemplary embodiments of the present invention, the fluorescent compound has high stability under a water-soluble condition to be easily stored for a long time and improve pH stability, and particularly, can be more efficiently used for labeling and dyeing of a target material to improve the fluorescent intensity even at a low concentration as compared with the conventional structure. Further, the fluorescent compound is excellent in optical stability and exhibits stable fluorescence in long-term dyeing, and is excellent in fluorescence intensity while being not accumulated in the body, and thus, can be easily dyed and imaged in vivo even in the use of a small amount as compared with the conventional dyes to be economically used.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 shows the absorbance fluorescent spectrum of the compounds 1-8 in accordance with embodiments of the present invention and a conventional dye.

FIG. 2 shows the optical characteristics of the compounds 1-8 in accordance with embodiments of the present invention and a conventional dye.

FIG. 3 shows the absorption characteristics of the compounds 1-11 in accordance with embodiments of the present invention and a conventional dye.

FIG. 4 shows the fluorescent intensity in the same molar concentration of the compounds 1-11 in accordance with embodiments of the present invention and a conventional dye.

FIG. 5 shows the fluorescent intensity in the same weight concentration of the compounds 1-11 in accordance with embodiments of the present invention and a conventional dye.

FIG. 6 shows the absorbance flourescent spectrum of the compounds 1-9 in accordance with embodiments of the present invention and a conventional dye.

FIG. 7 shows the optical characteristics of the compounds 1-9 in accordance with embodiments of the present invention and a conventional dye.

FIG. 8 shows the ratio of protein labeling of the compounds 1-9 in accordance with embodiments of the present invention and a conventional dye.

FIG. 9 shows the ratio of protein-labeling F/P of the compounds 1-9 in accordance with embodiments of the present invention and a conventional dye.

FIG. 10 shows the fluorescent intensity according to the ratio of protein labeling of the compounds 1-9 in accordance with embodiments of the present invention and a conventional dye.

FIG. 11 shows the absorption characteristics of the compounds 2-2 in accordance with embodiments of the present invention and a conventional dye in the same molar concentration.

FIG. 12 shows the fluorescent intensity in the same molar concentration of the compounds 2-2 in accordance with embodiments of the present invention and a conventional dye.

FIG. 13 shows the fluorescent intensity in the same weight concentration of the compounds 2-2 in accordance with embodiments of the present invention and a conventional dye.

FIG. 14 shows the ratio of protein labeling of the compounds 2-2 in accordance with embodiments of the present invention and a conventional dye.

FIG. 15. shows the graph of protein-labeling F/P of the compounds 2-2 in accordance with embodiments of the present invention and a conventional dye.

FIG. 16 shows the flourescent intensity according to the ratio of protein labeling of the compounds 2-2 in accordance with embodiments of the present invention and a conventional dye.

FIG. 17 shows the absorption intensity of the compounds 1-12 in accordance with embodiments of the present invention and a conventional dye in the same molar concentration.

FIG. 18 shows the fluorescent intensity in the same molar concentration of the compounds 1-12 in accordance with embodiments of the present invention and a conventional dye.

FIG. 19 shows the ratio of protein-labeling F/P of the compounds 1-12 in accordance with embodiments of the present invention and a conventional dye.

FIG. 20 shows the ratio of protein labeling of the compounds 1-12 in accordance with embodiments of the present invention and a conventional dye according to the reaction fold (F/P 1).

FIG. 21 shows the ratio of protein labeling of the compounds 1-12 in accordance with embodiments of the present invention and a conventional dye according to the reaction fold (F/P 2).

FIG. 22 shows the fluorescent intensity of the compounds 1-12 in accordance with embodiments of the present invention and a conventional dye according to the ratio of dye reaction.

FIG. 23 shows the fluorescent intensity of the compounds 1-12 in accordance with embodiments of the present invention and a conventional dye according to the ration of F/P 1.

FIG. 24 shows the fluorescent intensity of the compounds 1-12 in accordance with embodiments of the present invention and a conventional dye according to the ration of F/P 2.

### DETAILED DESCRIPTION OF EMBODIMENTS

A fluorescent compound in accordance with exemplary embodiments of the present invention is invented to improve the problem that the flourescent dye having aminosulfonic trizine has a lot of noise and the fluorescent effectiveness is low of that kind of dye. The present invention is a cyanine-based fluorescent dye in which the aminosulfonic triazine in introduced as a linker to solve the problem.

Hereinafter, preparation methods of a fluorescent compound and a surfactant compound in accordance with exemplary embodiments of the present invention and the fluorescence efficiency of the composition in accordance with exemplary embodiments of the present invention will be described in detail by using embodiments of the present invention.

Hereinafter, the present invention will be described in more detail through Examples of the present invention. However, the following Examples are not to limit the scope of the present invention and will be described to help in the understanding of the present invention.

Exemplary embodiments of the present invention may use a fluorescent compound represented by the following Chemical Formula 1.

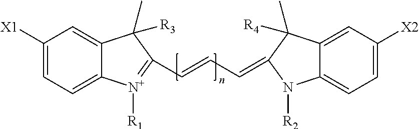

In Chemical Formula 1 above,

X1 and X2 are the same as or different from each other, and each independently selected from H, —SO₃⁻ and SO₃H,

R₁ and R₂ are the same as each other or each independently selected from C₁₋₇ alkyl, C₈₋₁₈ alkyl, —(CH₂)ₘSO₃⁻, —(CH₂)ₘSO₃H, and

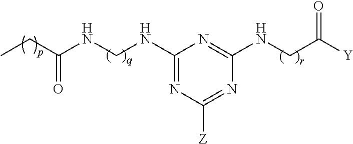

R₃ and R₄ are the same as or different from each other and each independently selected from C₁₋₇ alkyl, —(CH₂)ₘCOOZ and

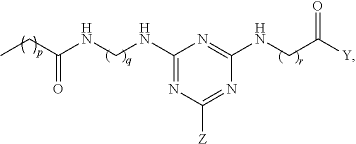

R₃ and R₄ are simultaneously not any one selected from —(CH₂)ₘCOOZ and

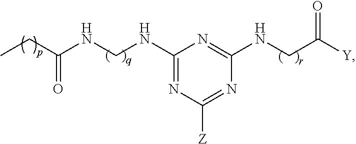

wherein,

n is an integer of 0 to 6,

m is an integer of 1 to 7,

p is an integer of 1 to 10,

q is an integer of 0 to 10,

r is an integer of 1 to 10, and

Z is OH or NH(CH₂)ₛSO₃H,

s is an integer of 1 to 7,

Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀₋₁₀ alkylamyl group, a vinylsulfonyl group, a vinylsulfonyl C₀₋₆ alkylaminyl group and an amino C₀₋₆ alkyl.

The compound of Chemical Formula 1 provided in exemplary embodiments of the present invention may be useful to detect the biomaterial by labeling the biomaterial, and the biomaterial may be selected from the group consisting of proteins, peptides, carbohydrates, sugars, fats, antibodies, proteoglycan, glycoprotein, and siRNA.

Further, when labeling the biomaterial, the fluorescent compound provided in exemplary embodiments of the present invention binds to at least one functional group selected from an amine group, a hydroxyl group, and a thiol group in the biomaterial to label the biomaterial.

The method for labeling the fluorescent compound represented by Chemical Formula 1 is performed by using a buffer selected from the group consisting of a phosphate buffer, a carbonate buffer, and a tris buffer, an organic solvent selected from the group consisting of dimethyl sulfoxide, dimethylformamide, methanol, ethanol and acetonitrile, or water as a solvent and reacting the biomaterial, nanoparticles, or organic compounds with the compound of Chemical Formula 1 at pH 5 to 12. The reaction may be carried out, for instance, for 30 minutes to 48 hours at a temperature of 20° C. to 80° C.

Most of biomaterials are dissolved in a predetermined buffer from a packaging unit, and in many cases, a separate buffer or pH is required to secure the stability of the biomaterials, and as a result, it is not easy to adjust the buffer or pH with a variable. The compound of Chemical Formula 1 according to exemplary embodiments of the present invention reacts reacting with proteins in various buffers, reaction temperatures, and pH conditions to express fluorescence and thus, is suitable to be used for labeling the biomaterials.

Exemplary preparation methods of the compounds included in Chemical Formula 1 will be described.

### Example 1: Synthesis of Initial Compound for Preparing Compounds in Accordance with Exemplary Embodiments of Present Invention

(1) Synthesis of Compound 3-1

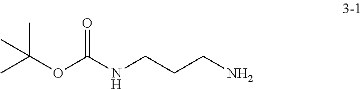

1,3-diaminopropane (20 g, 270 mmol, 7.96 eq) was dissolved in 70 ml of 1,4-dioxane. Di-tert-butyl dicarbonate (7.4 g, 33.9 mmol, 1 eq) was dissolved in 70 ml of 1,4-dioxane and then trickled in a 1,3-diaminopropane solution, and stirred at room temperature for a day and night and then dried under reduced pressure. The dried material was dissolved in distilled water and then filtered to extract the obtained filtrate with methylene chloride three times. An organic layer obtained after extraction was dried under reduced pressure to obtain a compound 3-1. (6 g, 91.5%)

R_{f}=0.4 (Silicagel, methylene chloride:methanol=8:1)

(2) Synthesis of Compound 3-2

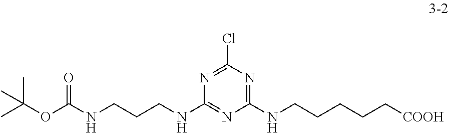 [SMILES: CC(C)(C)OC(=O)NCCCNc1nc(Cl)nc(NCCCCCC(=O)O)n1]

A compound 3-1 (5.1 g, 29.27 mmol, 1 eq) was dissolved in a mixed solution of 150 ml of acetone and 50 ml of distilled water and then stored at 4° C. or less. Cyanuric chloride (CNC) (5.4 g, 29.27 mmol, 1 eq) was fully dissolved in 150 ml of acetone and then added with 50 g of ice and dispersed at 4° C. or less. The compound 3-1 solution was trickled in a CNC solution, and then trickled in an aqueous solution of sodium hydrogencarbonate (fully dissolving 2.46 g carbonate in 50 ml of distilled water) and then, the reaction was performed at 4° C. or less for 2 hours. 6-aminohexanoic acid (1.42 g, 29.27 mmol, 1 eq) was dissolved in 50 ml of distilled water and then trickled in the reaction solution. The aqueous solution of sodium hydrogencarbonate was trickled and the reaction was performed at room temperature for 2 hours and then stirred at 4° C. for a day and night. The reaction solution was dried under reduced pressure and purified using silica gel chromatography to obtain a compound 3-2. (9 g, 73.8%)

R_{f}=0.7 (Silicagel, methylene chloride:methanol=8:1)

LC/MS, calculated value of C₁₇H₂₉ClN₆O₄ 416.91, measured value of 415.2

(3) Synthesis of Compound 3-3

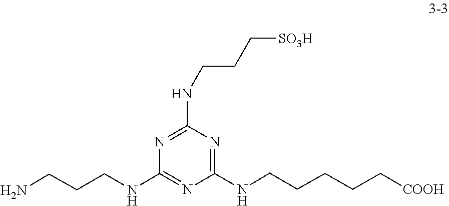 [SMILES: NCCCNc1nc(NCCCCCC(=O)O)nc(NCCCS(=O)(=O)O)n1]

The compound 3-2(4 g, 9.61 mmol, 1 eq) and 3-Amino-1-propanesulfonic acid) (1.6 g, 11.53 mmol, 1.2 eq) were fully dissolved in 6.7 ml of imethylformamide (DMF) and then added with 40 ml of distilled water. Thereafter 2 ml of 30% sodium hydroxide was added and then stirred in 4 hours at 100° C. and the reaction-solution was lyophilized.

Thereafter, 40 ml of a 6 N aqueous hydrochloric acid solution was added, and then the solution was reacted for 2 hours at room temperature. The reaction solution was lyophilized and subjected to a reverse phase column to obtain a compound 3-3. (1.6 g, 40%)

R_{f}=0.23 (Silicagel, methylene chloride:methanol=8:1)

LC/MS, calculated value of C₁₅H₂₉N₇O₅S 298.35, measured value of 419.50

### Example 2: Synthesis of Initial Compound 1-1 for Preparing Compounds in Accordance with Exemplary Embodiments of the Present Invention

(1) Synthesis of Compound 4-1

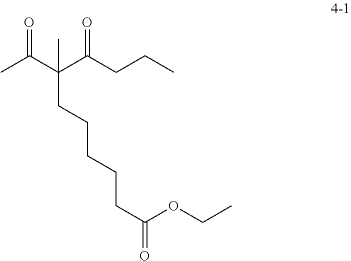 [SMILES: CCCC(=O)C(C)(CCCCCC(=O)OCC)C(C)=O]

Ethyl 2-methyl acetoacetate (29.2 ml, 0.203 mol, 1 eq), a 21% sodium ethoride solution (64 ml, 0.816 mol, 4 eq), ethyl 6-bromohexanoate (34 ml, 0.192 mol, 1 eq), and ethanol (200 ml) were added and then refluxed at 120° C. for 12 hours. Thereafter, the solvent was extracted by neutralizing pH using 1 M hydrochloric acid and then using chloroform and distilled water. The extracted solvent was dried under reduced pressure and purified using normal chromatography to obtain a compound 4-1. (36.8 g, 63.4%)

R_{f}=0.34 (Silicagel, Hexane/ethyl acetate=10:1 v/v)

(2) Synthesis of Compound 4-2

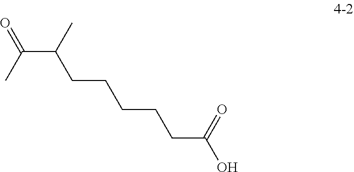

Sodium hydroxide (6.2 g, 0.170 mol, 3.5 eq), methanol (47.2 ml), and distilled water (15.6 ml) were added to the compound 4-1 (13.7 g, 0.0486 mol, 1 eq) and then refluxed at 50° C. for 12 hours. Thereafter, the solvent was dried under reduced pressure and then extracted by adjusting pH to 1 using 1 M hydrochloric acid and using ethyl acetate, and then dried under reduced pressure to obtain a compound 4-2. (8.17 g, 90.7%)

R_{f}=0.05 (Silicagel, Hexane/ethyl acetate=10:1 v/v)

(3) Synthesis of Compound 4-3

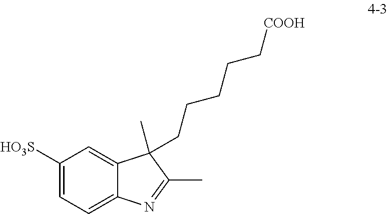 [SMILES: CC1=Nc2ccc(S(=O)(=O)O)cc2C1(C)CCCCCC(=O)O]

p-hydrazinobenzensulfonic acid hemihydrate (8.25 g, 0.0438 mol, 1 eq) and acetic acid were added to the compound 4-2 (8.165 g, 0.0438 mol, 1 eq) and then refluxed at 120° C. for 5 hours. The mixture was dried under reduced pressure and then purified using normal chromatography and dried under reduced pressure to obtain a compound 4-3. (12.6 g, 84.8%)

R_{f}=0.51 (Silicagel, isobutanol/n-propanol/ethyl acetate/water 2:4:1:3 v/v/v/v)

(4) Synthesis of Compound 4-4

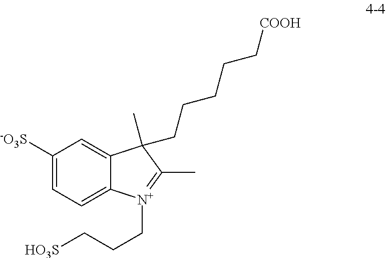

Sodium acetic acid (4.16 g, 0.061 mol, 1.65 eq), 1,3-propane sultone (21.3 ml, 0.243 mol, 6.57 eq), and acetonitrile (24.8 ml) were added to the compound 4-3 (12.57 g, 0.037 mol, 1 eq) and then refluxed at 110° C. for 5 hours. Thereafter, the mixture was dried under reduced pressure and then purified using reverse phase chromatography and dried under reduced pressure to obtain a compound 4-4. (12 g, 70.6%)

R_{f}=0.3 (Silicagel, isobutanol/n-propanol/ethyl acetate/water 2:4:1:3 v/v/v/v)

(5) Synthesis of Compound 4-5

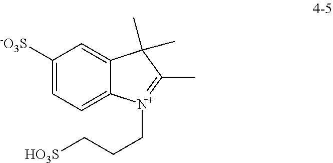

Sodium acetate (17.87 g, 0.216 mol, 1.2 eq), 1,3-propane sultone (70.5 ml, 0.8 mol, 4.5 eq), and acetonitrile (42 ml) were added to the compound 4-1 (50 g, 0.18 mol, 1 eq). Thereafter, the mixture was refluxed at 110° C. for 12 hours and then particles were captured using ethyl acetate and dried to obtain a compound 4-5. (61 g, 94%)

R_{f}=0.3 (Silicagel, isobutanol/n-propanol/ethyl acetate/water 2:4:1:3 v/v/v/v)

(6) Synthesis of Compound 4-6

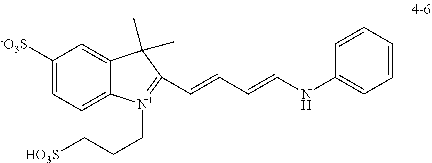

Malonaldehyde dianilide hydrochloride (42.9 g, 0.166 mol, 1 eq), triethylamine (2.3 ml, 0.016 mol, 0.1 eq), and acetic acid (551 ml) were added to the compound 4-5 (60 g, 0.166 mol, 1 eq) and then heated and refluxed at 140° C. Thereafter, the particles were precipitated using ethyl acetate and then dried. The compound was purified using normal chromatography and then dried under reduced pressure to obtain a compound 4-6. (7.5 g, 8.5%)

R_{f}=0.55 (Silicagel, isobutanol/n-propanol/ethyl acetate/water 2:4:1:3 v/v/v/v)

(7) Synthesis of Compound 1-1

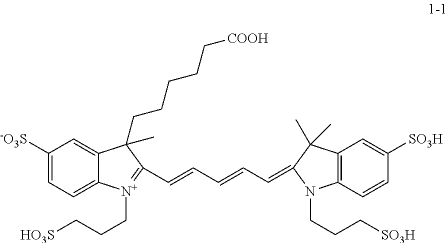

The compound 4-4 (6.5 g, 0.014 mol, 1 eq) and the compound 4-6 (7.5 g, 0.014 mol, 1 eq) were added to a mixed solution of triethylamine (16.6 ml, 0.12 mol, 8.5 eq), anhydrous acetic acid (7.3 ml), and DMF (75 ml) and then reacted at room temperature for 1 hour. Thereafter, the particles were precipitated using ethyl acetate and then dried. The compound was purified using normal chromatography and then dried under reduced pressure to obtain a compound 1-1.

(250 mg, 2%)

R_{f}=0.4 (Silicagel, isobutanol/n-propanol/ethyl acetate/water 2:4:1:3 v/v/v/v)

LC/MS, calculated value of C₃₆H₄₄N₂Na₂O₁₄S₄ 902.98, measured value of 901

### Example 3: Synthesis of Compound 1-2 as Compound in Accordance with Exemplary Embodiments of the Present Invention

(1) Synthesis of Compound 2-1

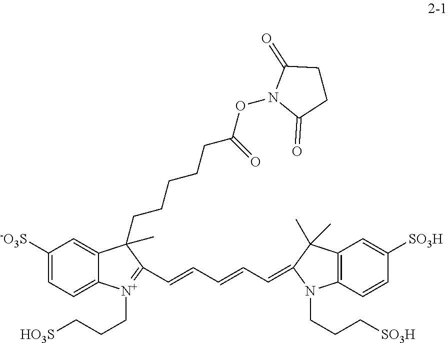

The compound 1-1 (100 mg, 0.1165 mmol, 1 eq), TSTU (77 mg, 0.2563 mmol, 2.2 eq), and triethylamine (125 ul, 0.897 mmol, 7.7 eq) were added to 10 mL of DMF and reacted at room temperature for 40 minutes. Solid particles generated after reaction were filtered. The solid particles were washed with ethyl acetate 2 to 3 times to obtain a compound 2-1. (111 mg, 100%)

R_{f}=0.44 (Silicagel, isobutanol/n-propanol/ethyl acetate/water 2:4:1:3 v/v/v/v)

LC/MS, calculated value of C₄₀H₄₉N₃O₁₆S₄ 956.09, measured value of 954

(2) Synthesis of Compound 1-2

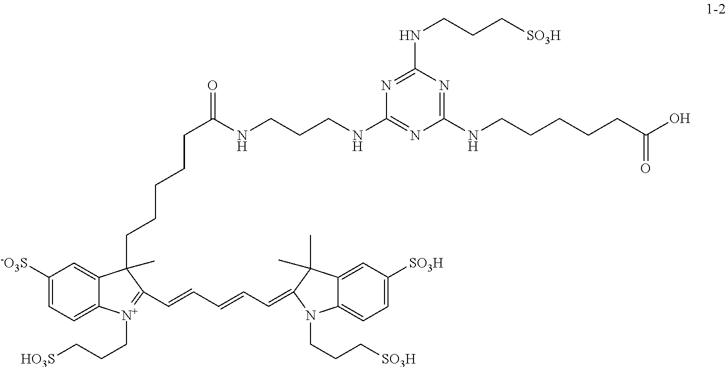

The compound 2-1 (55.4 mg, 0.058 mmol, 1 eq) was fully dissolved in 4 mL of DMF. The compound 3-3 (73 mg, 0.174 mmol, 3 eq) was fully dissolved in 1 ml of DMF and then added to a compound 2-1 solution and then added with Wheeinig base (50.5 □l, 10 eq) and stirred day and night at room temperature. After the reaction was confirmed, particles were produced by adding ether and filtered and dried. The obtained material was purified using reverse phase chromatography and then dried under reduced pressure to obtain a compound 1-2.

(14 mg, 19.2%)

R_{f}=0.17 (Silicagel, isobutanol/n-propanol/ethyl acetate/water 2:4:1:3 v/v/v/v)

LC/MS, calculated value of C₅₁H₇₃N₉O₁₈S₅ 1260.49, measured value of 1260.49

### Example 4: Synthetic Method of Compound 2-2 as Compound in Accordance with Exemplary Embodiments of the Present Invention

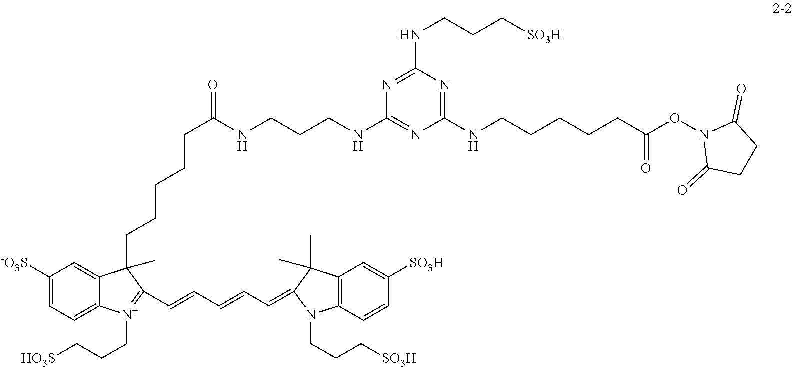

The compound 1-2 (14 mg, 0.011 mmol, 1 eq), TSTU (10 mg, 0.033 mmol, 3 eq) and triethylamine (7.7 ul, 0.055 mmol, 5 eq) were added in 2 ml of DMF and then reacted in 1 hour at room temperature. After the reaction was confirmed, particles were produced and filtered. By washing the particles in ethylacetate 2 it 3 times and then dried under reduced pressure to obtain a compound 2-2. (9.63 mg, 64.6%)

R_{f}=0.22 (Silicagel, isobutanol/n-propanol/ethyl acetate/water 2:4:1:3 v/v/v/v)

LC/MS, calculated value of C₅₅H₇₆N₁₀O₂₀S₅ 1357.56, measured value of 1356.38

### Example 5-9: Synthetic Method of Compounds 1-3, 1-4, 1-5, 1-6 and 1-7 as the Compounds in Accordance with Exemplary Embodiments of the Present Invention

As the similar manner as described in Examples 1 to 4, Examples 5 to 9 were conducted.

### Example 5. Synthesis of Compound 1-3

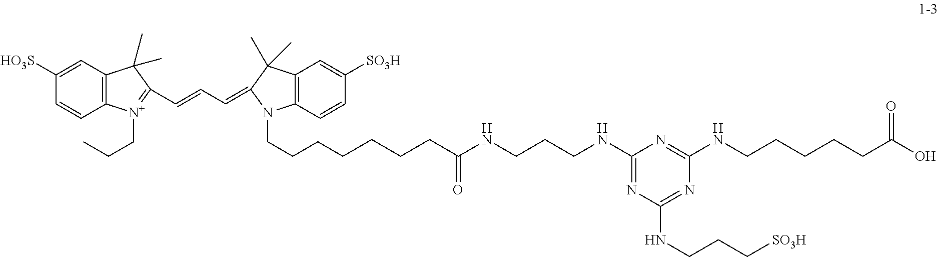 [SMILES: CCC[N+]1=C(/C=C/C=C2/N(CCCCCCCC(=O)NCCCNc3nc(NCCCCCC(=O)O)nc(NCCCS(=O)(=O)O)n3)c3ccc(S(=O)(=O)O)cc3C2(C)C)C(C)(C)c2cc(S(=O)(=O)O)ccc21]

(113 mg, 64.4%)

R_{f}=0.45 (Silicagel, isobutanol/n-propanol/ethyl acetate/water 2:4:1:3 v/v/v/v)

LC/MS, calculated value of C₄₉H₇₁N₉O₁₂S₃ 1074.34, measured value of 1071.8

### Example 6. Synthesis of Compound 1-4

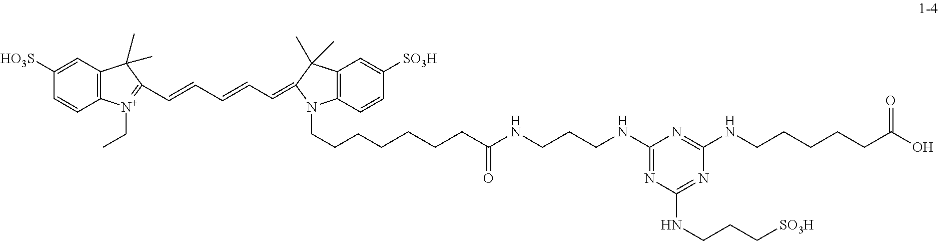 [SMILES: CC[N+]1=C(/C=C/C=C/C=C2/N(CCCCCCCC(=O)NCCCNc3nc(NCCCCCC(=O)O)nc(NCCCS(=O)(=O)O)n3)c3ccc(S(=O)(=O)O)cc3C2(C)C)C(C)(C)c2cc(S(=O)(=O)O)ccc21]

(120 mg, 80.0%)

R_{f}=0.6 (Silicagel, isobutanol/n-propanol/ethyl acetate/water 2:4:1:3 v/v/v/v)

LC/MS, calculated value of C₅₀H₇₁N₉O₁₂S₃ 1086.35, measured value of 1084.7

### Example 7. Synthesis of Compound 1-5

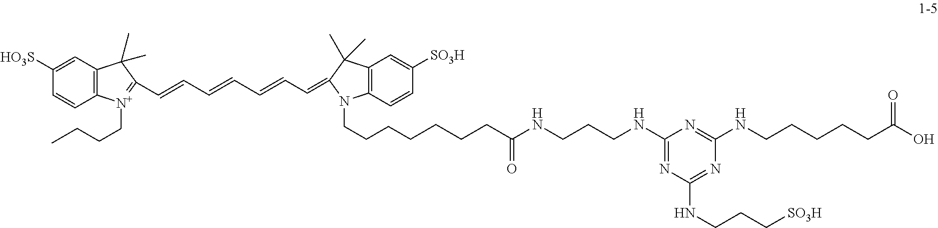 [SMILES: CCCC[N+]1=C(/C=C/C=C/C=C/C=C2/N(CCCCCCCC(=O)NCCCNc3nc(NCCCCCC(=O)O)nc(NCCCS(=O)(=O)O)n3)c3ccc(S(=O)(=O)O)cc3C2(C)C)C(C)(C)c2cc(S(=O)(=O)O)ccc21]

(110 mg, 70.0%)

R_{f}=0.55 (Silicagel, isobutanol/n-propanol/ethyl acetate/water 2:4:1:3 v/v/v/v)

LC/MS, calculated value of C₅₂H₇₃N₉O₁₂S₃ 1112.39, measured value of 1181.8

### Example 8. Synthesis of Compound 1-6

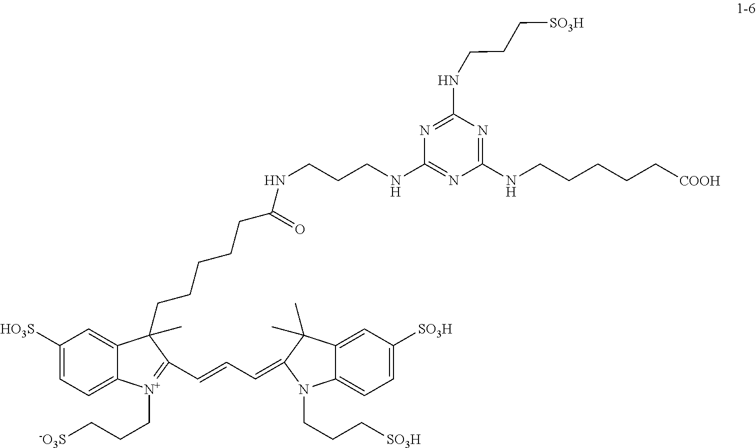

(102 mg, 68.9%)

R_{f}=0.1 (Silicagel, isobutanol/n-propanol/ethyl acetate/water 2:4:1:3 v/v/v/v)

LC/MS, calculated value of C₄₉H₇₁N₉O₁₈S₅ 1234.45, measured value of 1136.18

### Example 9. Synthesis of Compound 1-7

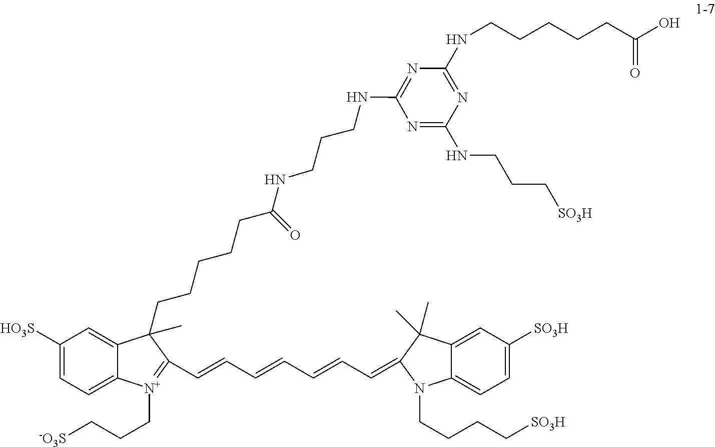

(45 mg, 31.3%)

R_{f}=0.125 (Silicagel, isobutanol/n-propanol/ethyl acetate/water 2:4:1:3 v/v/v/v)

LC/MS, calculated value of C₅₄H₇₇N₉O₁₈S₅ 1300.56, measured value of 1302.94

### Example 10-14: Synthetic Method of Compounds 1-8, 1-9, 1-10, 1-11 and 1-12 as the Compounds in Accordance with Exemplary Embodiments of the Present Invention

As the similar manner as described in Examples 1 to 4, Examples 10 to 14 were conducted.

### Example 10. Synthesis of Compound 1-8

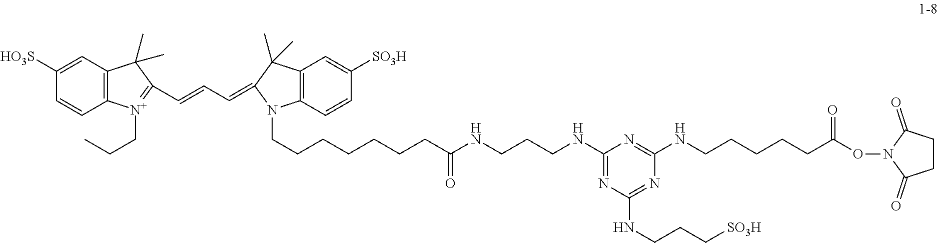 [SMILES: CCC[N+]1=C(/C=C/C=C2/N(CCCCCCCC(=O)NCCCNc3nc(NCCCCCC(=O)ON4C(=O)CCC4=O)nc(NCCCS(=O)(=O)O)n3)c3ccc(S(=O)(=O)O)cc3C2(C)C)C(C)(C)c2cc(S(=O)(=O)O)ccc21]

(113 mg, 64.4%)

R_{f}=0.45 (Silicagel, isobutanol/n-propanol/ethyl acetate/water 2:4:1:3 v/v/v/v)

LC/MS, calculated value of C₅₃H₇₄N₁₀O₁₄S₃ 1171.41, measured value of 1168

### Example 11. Synthesis of Compound 1-9

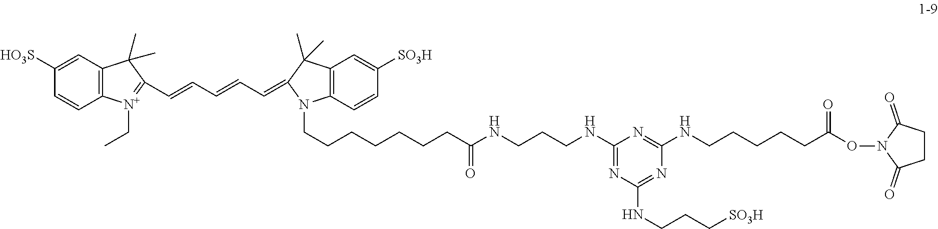 [SMILES: CC[N+]1=C(/C=C/C=C/C=C2/N(CCCCCCCC(=O)NCCCNc3nc(NCCCCCC(=O)ON4C(=O)CCC4=O)nc(NCCCS(=O)(=O)O)n3)c3ccc(S(=O)(=O)O)cc3C2(C)C)C(C)(C)c2cc(S(=O)(=O)O)ccc21]

(120 mg, 80.0%)

R_{f}=0.6 (Silicagel, isobutanol/n-propanol/ethyl acetate/water 2:4:1:3 v/v/v/v)

LC/MS, calculated value of C₅₄H₇₄N₁₀O₁₄S₃ 1183.42, measured value of 1181.6

### Example 12. Synthesis of Compound 1-10

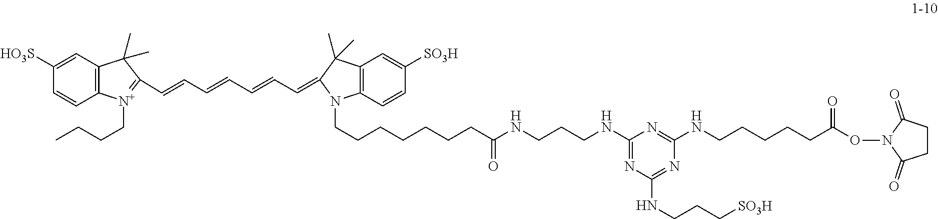 [SMILES: CCCC[N+]1=C(/C=C/C=C/C=C/C=C2/N(CCCCCCCC(=O)NCCCNc3nc(NCCCCCC(=O)ON4C(=O)CCC4=O)nc(NCCCS(=O)(=O)O)n3)c3ccc(S(=O)(=O)O)cc3C2(C)C)C(C)(C)c2cc(S(=O)(=O)O)ccc21]

(110 mg, 70.0%)

R_{f}=0.55 (Silicagel, isobutanol/n-propanol/ethyl acetate/water 2:4:1:3 v/v/v/v)

LC/MS, calculated value of C₅₆H₇₆N₁₀O₁₄S₃ 1209.46, measured value of 1206

### Example 13. Synthesis of Compound 1-11

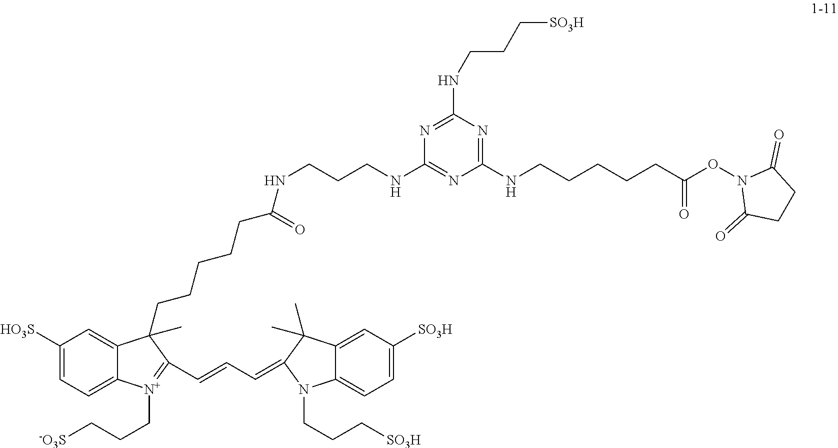

(102 mg, 68.9%)

R_{f}=0.1 (Silicagel, isobutanol/n-propanol/ethyl acetate/water 2:4:1:3 v/v/v/v)

LC/MS, calculated value of C₅₃H₇₄N₁₀O₂₀S₅ 1331.53, measured value of 1333.43

### Example 14. Synthesis of Compound 1-12

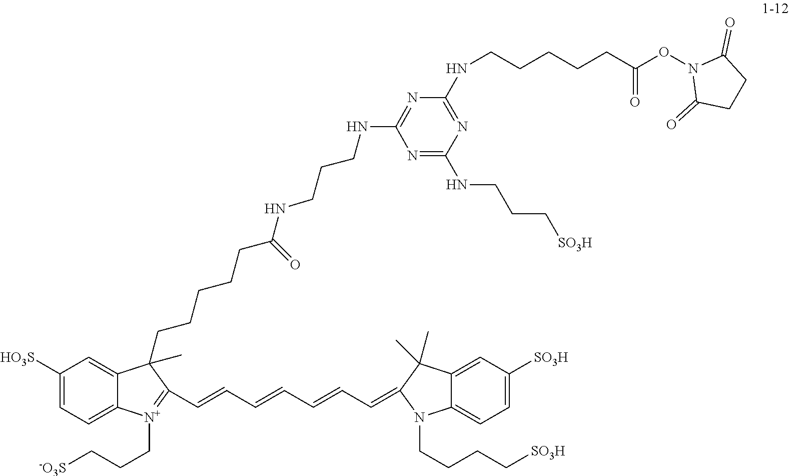

(45 mg, 31.3%)

R_{f}=0.125 (Silicagel, isobutanol/n-propanol/ethyl acetate/water 2:4:1:3 v/v/v/v)

LC/MS, calculated value of C₅₈H₈₀N₁₀O₂₀S₅ 1397.63, measured value of 1399.24

Fluorescent values at the same absorption wavelength of the compounds of exemplary embodiments of the present invention and the conventional compounds not included in exemplary embodiments of the present invention were compared through Comparative Examples. Thereafter, the usability of the present invention in detection biomaterials was validated.

### Comparative Example 1. Comparison of Optical Characteristics of Compound 1-8

(1) Comparison of the Characteristics of Absorption and Flourescence

The absorption and fluorescence characteristics of the compound 1-8 in accordance with the present invention and a conventional dye (Flamma® 552 NHS ester) were analyzed and verified by the analysis

Firstly, the said two fluorescent dyes were dissolved in DMF and the stock solution were prepared (10 mg/ml) and the pH of the stock was maintained 7.4 and diluted by 10 mM phosphate buffered saline (1×PBS). Then the analysis were proceeded. The absorption were measured by Cary 3500 UV-Vis photometer and the fluorescence were measured by LS 55 fluorescence spectrometer (PerkinElmer).

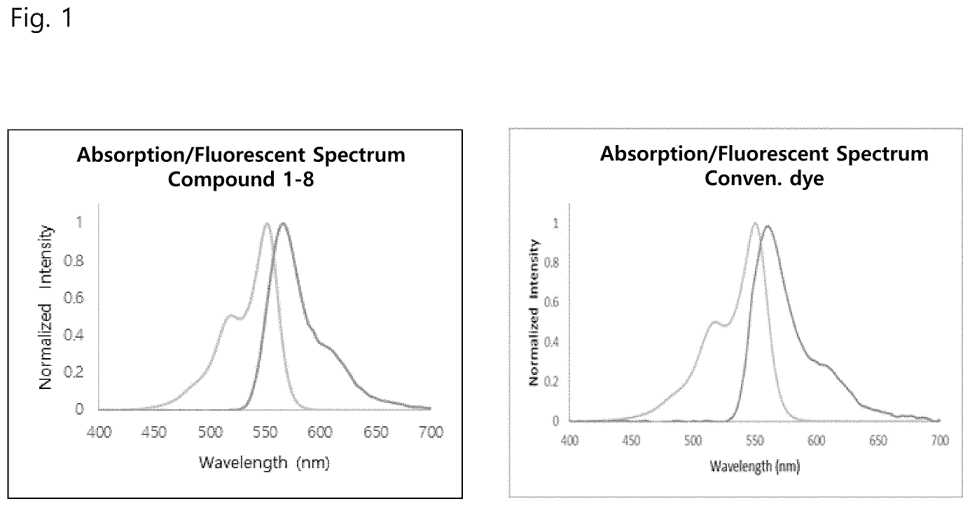

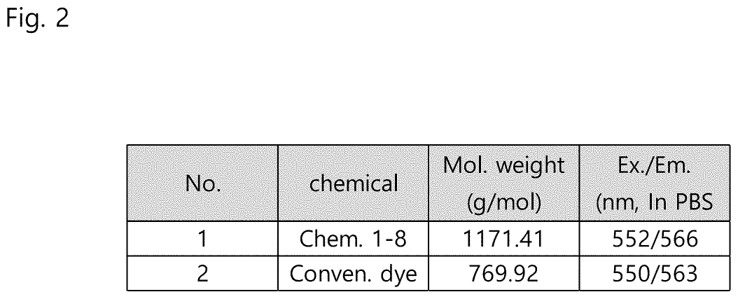

FIG. 1 shows the absorption and fluorescence spectrum of the two dyes, FIG. 2 represents the optical characteristics of the dyes. FIG. 1 and FIG. 2 verify that the compound 1-8 in accordance with the present invention has similar optical characteristics as those of the conventional dye.

### Comparative Example 2. Estimation of the Optical Characteristics of Compound 1-11

(1) Comparison of Absorption Characteristics

The absorption characteristics of the compound 1-11 in the present invention and a conventional dye (Invitrogen, Alexa Flour™ 555 NHS ester) were compared.

Each stock solution were prepared by adding the said two dyes in DMF. The concentration of the stock solutions were same each other as 10 mg/ml. For comparing the absorption characteristics in the same molar concentration, the stock solutions were diluted by 10 mM PBS until the concentrations of each stock reach 10 uM and the absorption characteristics were analyzed by spectrophotometer (Agilent, Cary 3500 UV-Vis).

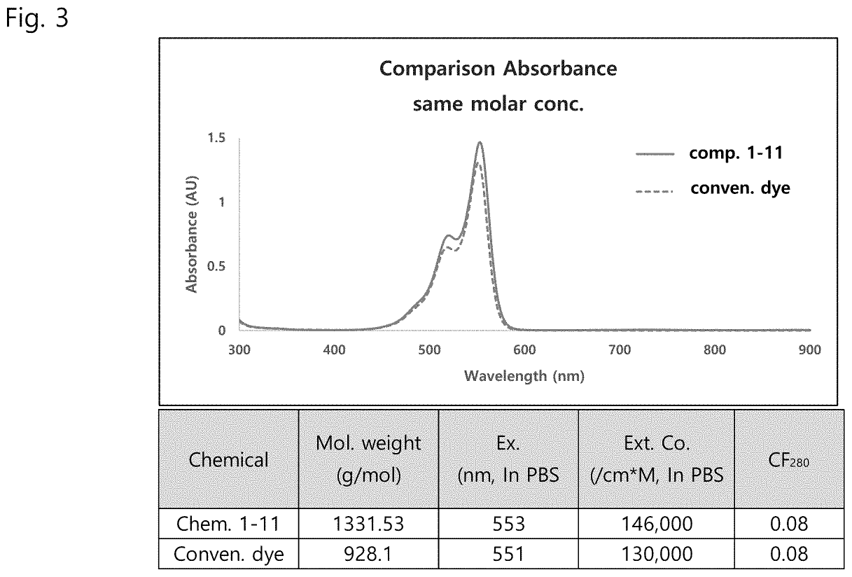

The result of the comparison were showed in FIG. 3. FIG. 3 shows that the absorption intensity and molar extinction coefficient of the compound 1-11 were relatively higher than those of the conventional dye.

(2) The Comparison of Fluorescent Characteristics and Intensity

The fluorescent characteristics and intensity of the compound 1-11 and the conventional dye were compared.

Each stock solution was prepared by adding the said two dyes in DMF. The concentrations of the stock solutions were same each other as 10 mg/ml.

Firstly, to compare the fluorescent intensity at the same molar concentration, the stock solutions were diluted by 1×PBS until the concentrations reach 0.013 uM and then the fluorescence were measured at the extinction wavelength of the dyes.

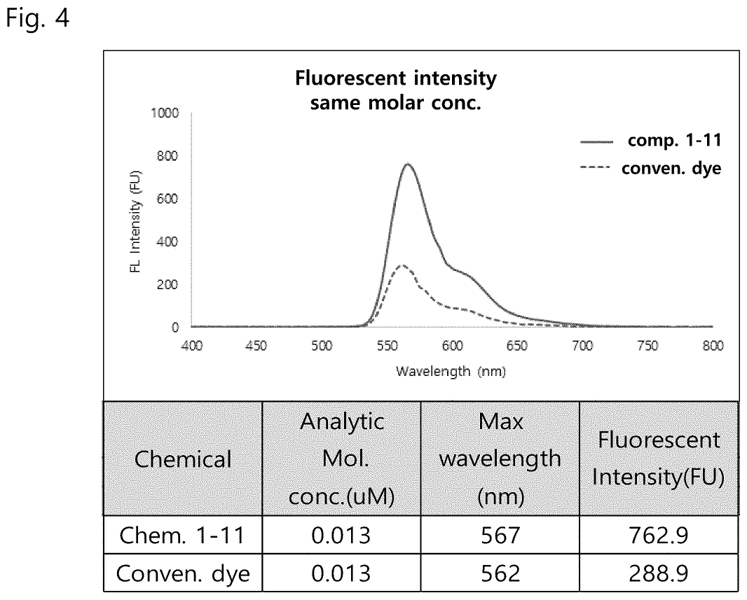

The fluorescence was measured by LS 55 Fluorescence spectrometer (PerkinElmer) and the results were showed in FIG. 4.

Thereafter, to compare the fluorescent intensity of each dye in the same weight concentration, the stock solutions of the two dyes were diluted by 1×PBS until the concentrations reach 0.013 ug/ml and the fluorescence were measured.

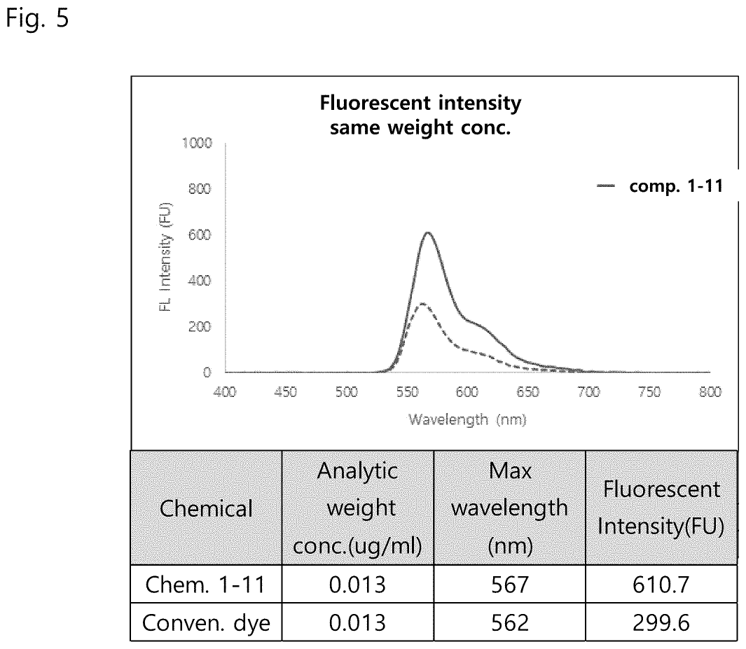

The results were presented in FIG. 5.

FIG. 4 and FIG. 5 show that the fluorescent intensity of the compound 1-11 is higher than the conventional dye both at the same molar concentration and at the same weight concentration. The maximum fluorescent wavelengths of the compound 1-11 and the conventional dye in the solvent 1×PBS are 567 nm and 562 nm respectively.

### Comparative Example 3. Estimation of the Optical Characteristics of Compound 1-9

(1) Comparison of the Characteristics of Absorption and Fluorescence

The absorption and fluorescence characteristics of the compound 1-8 in accordance with the present invention and a conventional dye (Flamma® 648 NHS ester) were analyzed and verified by the analysis

Firstly, the said two fluorescent dyes were dissolved in DMF, and the stock solution was prepared (10 mg/ml). The pH of the stock solution was maintained 7.4 and diluted by 10 mM phosphate buffered saline (1×PBS). Then analysis was carried out. The absorption was measured by Cary 3500 UV-Vis photometer, and the fluorescence was measured by LS 55 fluorescence spectrometer (PerkinElmer).

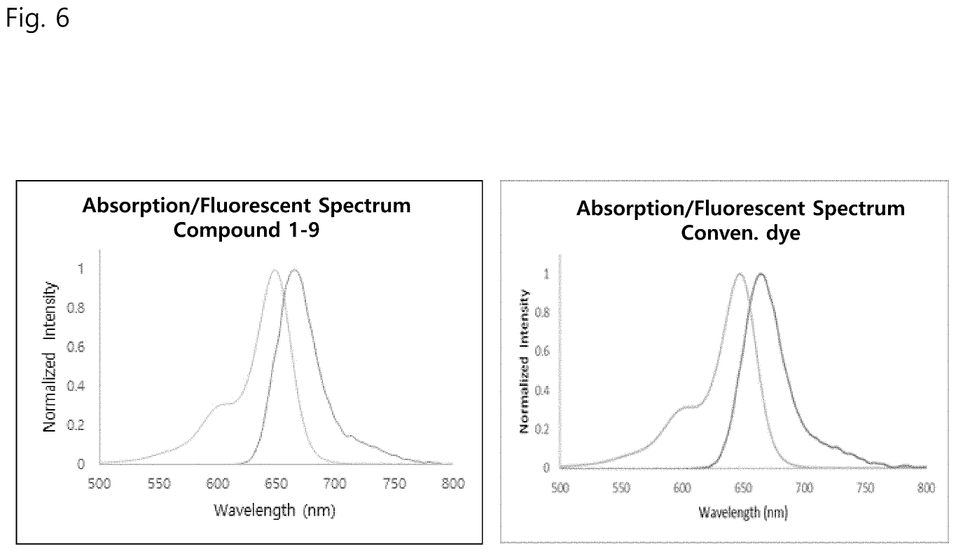

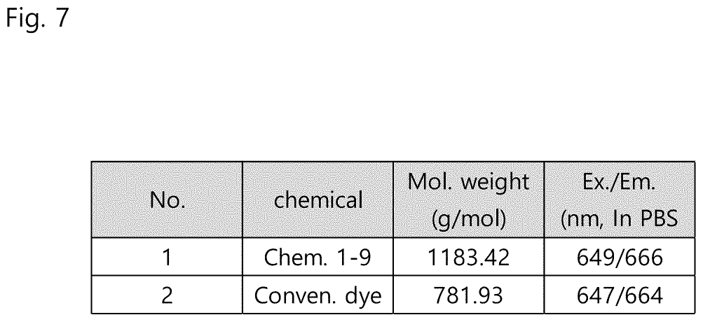

FIG. 6 shows the absorption and fluorescence spectrum of the two dyes, and FIG. 7 represents the optical characteristics of the dyes. FIG. 6 and FIG. 7 verify that the compound 1-8 in accordance with the present invention has similar optical characteristics as those of the conventional dye.

(2) Comparison of Performance after Protein Labeling

a. Comparison of the Ratio of Protein Labeling of Each Compound According to the Reaction Ratio

After antibody-labeling (Invitrogen, Goat anti Rabbit IgG H+L Secondary Ab, 150 kDa) of the compound 1-9 in accordance with the present invention and a conventional dye (Flamma® 648 NHS ester), the labeling ratio (F/P molar ratio) were measured.

Before the labeling, the compound 1-9 and the conventional dye were dissolved in DMF, and the stock solution of 10 mg/ml was prepared. Thereafter the dyes were reacted with 0.1 mg of the antibody by each reaction ratio (2, 5, 15, 25, 33 Fold). The reaction buffer was prepared with the final pH of 8.3˜8.5, and the final concentration of the antibody was 2 mg/ml. It was reacted for 1 hour in dark environment at room temperature through stirring, and the reaction particles were separated and obtained through the column filled with Sephadex G-25 resin(Cytiva). The resin was used in a buffer equilibrium with 1×PBS.

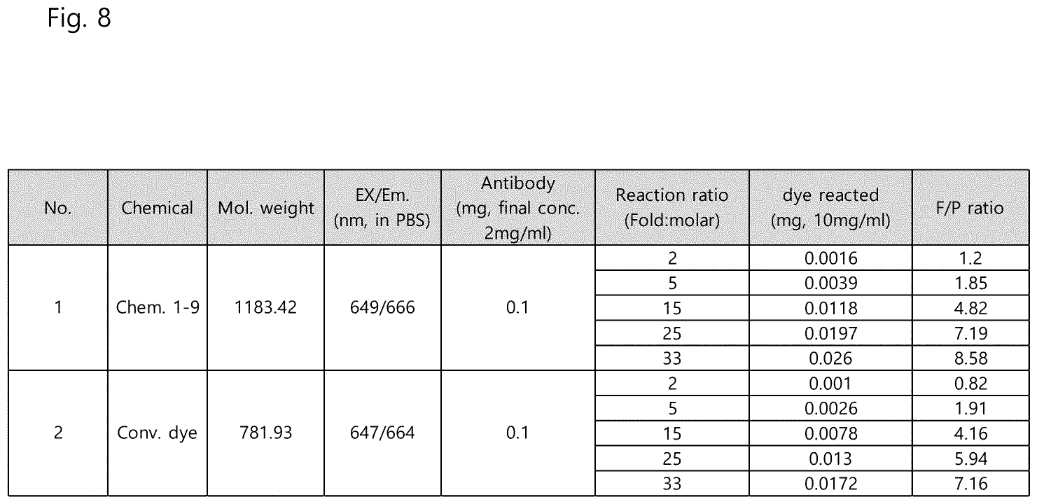

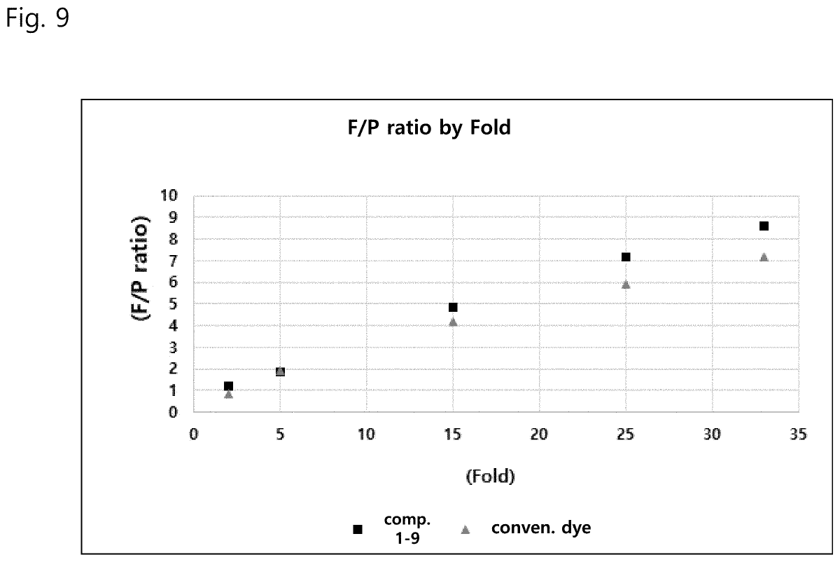

The fluorescent intensity of each reaction particle was measured at the wavelength of 280, 648 nm respectively (Agilent, Cary 3500 UV-Vis spectrophotometer), and the labeling ratio was calculated through a universal formula. The results were shown in FIG. 8 and FIG. 9.

The F/P ratio, which is the standard of the conventional dye (Flamma® 648 NHS ester), was calculated by applying Extinction coefficient 250,000/M·cm, CF₂₈₀ 0.03 which is close to analytic and measured values of the compound 1-9 and the conventional dye. From the results of the experiments, the labeling ratio by reaction ratio of the compound 1-9 was analyzed and found higher than that of the conventional dye.

b. Comparison of Fluorescent Intensity According to Chemical Labeling

The fluorescent intensity by the reaction ratio in the said comparison experiment 3-(2)-a and the fluorescent intensities of the two dyes by the labeling ratio were compared.

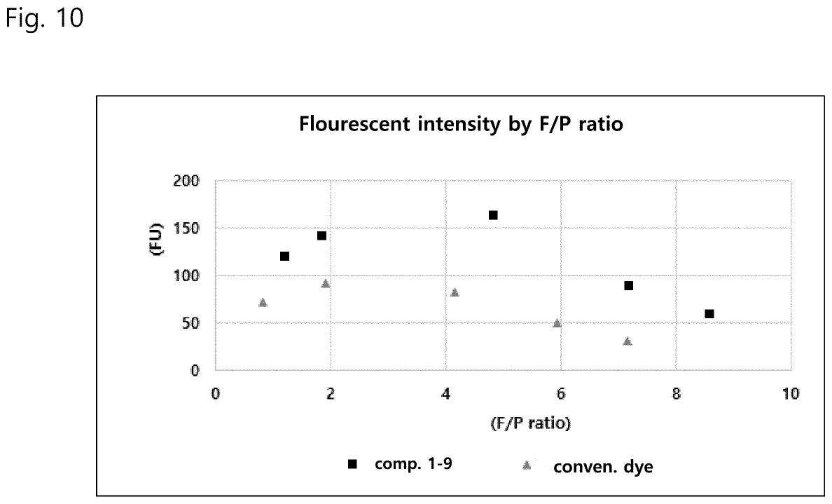

FIG. 10 shows the results and the fluorescent intensities were measured by the said PerkinElmer device.

From FIG. 9, it is clear that the fluorescent intensity of the compound 1-9 is higher than that of the conventional dye in all of the labeling ratio (reaction ratio), especially when we compare the intensity of pseudo-labeling ratio (ratio of about 1, 2, 7) between the objects the fluorescent intensity of the compound 1-9 to the antibody is more excellent than that of the conventional dye to the antibody.

### Comparative Example 4. Estimation of the Optical Characteristics of Compound 2-2

(1) Comparison of Absorption Characteristics

The absorption characteristics of the compound 2-2 in the present invention and a conventional dye (Invitrogen, Alexa Flour™ 647 NHS ester) were compared.

Each stock solution was prepared by adding the said two dyes in DMF. The concentrations of the stock solutions were the same as 10 mg/ml.

First, to compare the absorption characteristics at the same molar concentration, the stock solutions were diluted by pH 7.4 1×PBS until the concentrations reach 5.31 uM and then the absorbances were analyzed by Agilent, Cary 3500 UV-Vis spectrophotometer.

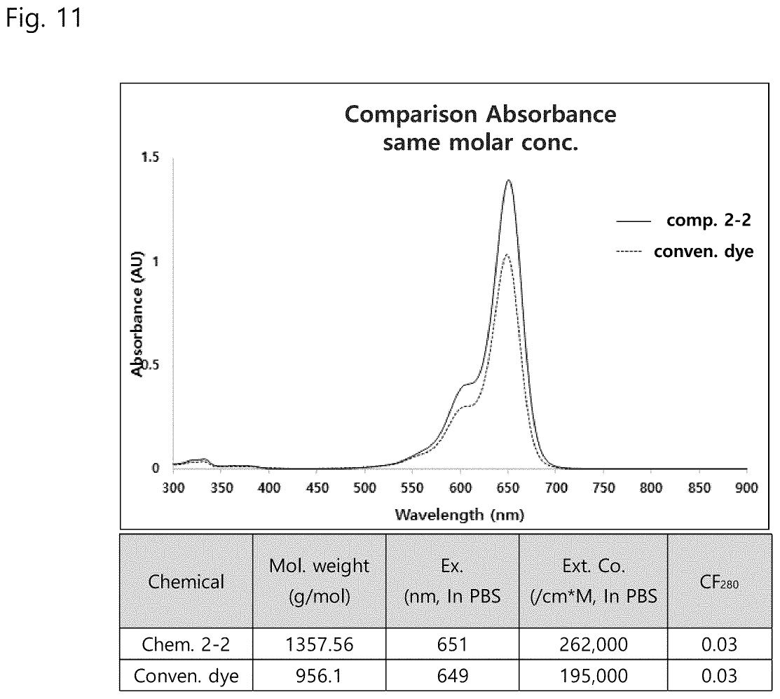

FIG. 11 shows the absorption characteristics of the two dyes. The absorption intensity and the molar absorbance index of the compound 2-2 were relatively higher than those of the conventional dye.

(2) The Comparison of Fluorescent Characteristics and Intensity

The fluorescent characteristics and intensity of the compound 2-2 and the conventional dye were compared.

Each stock solution was prepared by adding the said two dyes in DMF. The concentrations of the stock solutions were the same as 10 mg/ml.

First, to compare the fluorescent intensity at the same molar concentration, the stock solutions were diluted by 1×PBS until the concentrations reach 0.0221 uM and then the fluorescence were measured at the extinction wavelength of 650 nm.

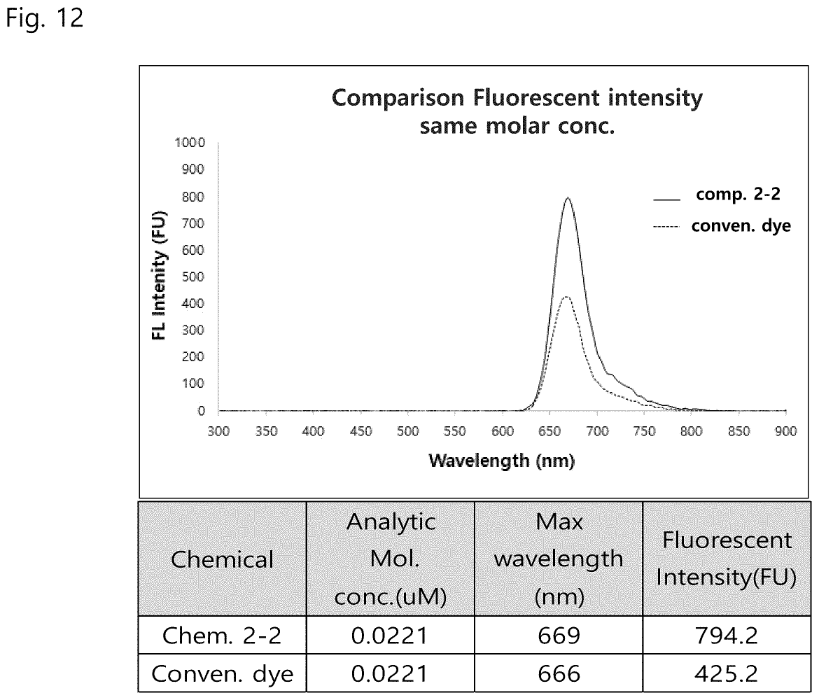

The fluorescence was measured by LS 55 Fluorescence spectrometer (PerkinElmer), and the results were shown in FIG. 12.

Thereafter, to compare the fluorescent intensity of each dye at the same weight concentration, the stock solutions of the two dyes were diluted by 1×PBS until the concentrations reach 0.0417 ug/ml and the fluorescence were measured.

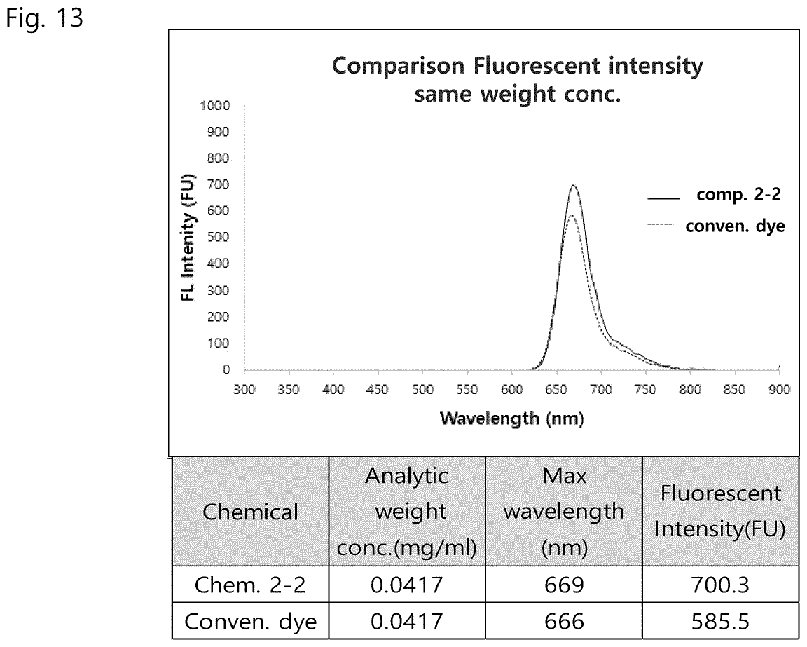

The results were shown in FIG. 13.

The results show that the fluorescent intensity of the compound 2-2 is higher than the conventional dye both in the same molar concentration and in the same weight concentration.

(3) Comparison of Performance after Protein Labeling

a. Comparison of the Ratio of Protein Labeling of Each Compound According to the Reaction Ratio

After antibody-labeling (Invitrogen, Goat anti Rabbit IgG H+L Secondary Ab, 150 kDa) of the compound 2-2 in accordance with the present invention and a conventional dye (Flamma® 647 NHS ester), the labeling ratio (F/P molar ratio) were measured and compared.

Before the labeling, the compound 2-2 and the conventional dye were dissolved in DMF and the stock solution of 10 mg/ml was prepared. Thereafter the dyes were reacted with 0.1 mg of the antibody by each reaction ratio (2, 5, 15, 25, 33 Fold). The reaction buffer was prepared with the final pH of 8.3˜8.5, and the final concentration of the antibody was 2 mg/ml. The reaction was executed in 1 hour in dark environment at room temperature through stirring, and the reaction particles were separated and obtained through the column filled with Sephadex G-25 resin(Cytiva). The resin was used in a buffer equilibrium with 1×PBS.

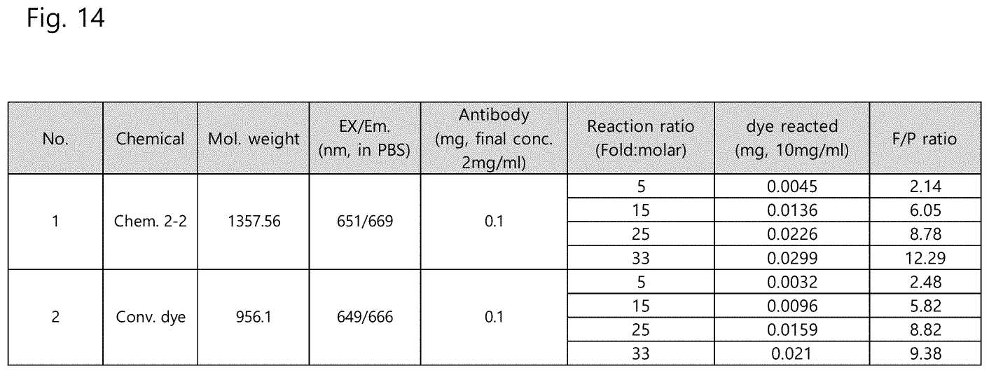

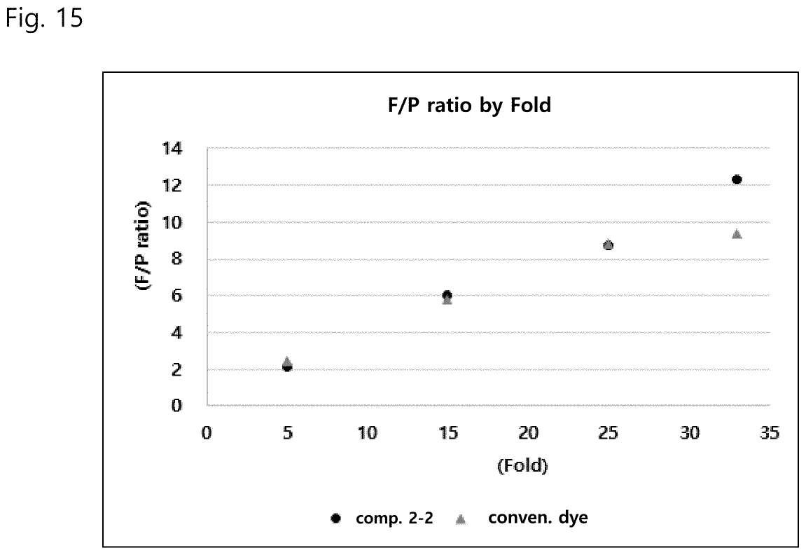

The fluorescent intensities of the reaction particles were measured at the wavelengths of 280, 648 nm respectively (Agilent, Cary 3500 UV-Vis spectrophotometer), and the labeling ratio was calculated through a universal formula. The results were shown in FIG. 14 and FIG. 15.

The F/P ratio, which is the standard of the conventional dye (Flamma® 647 NHS ester), was calculated by applying Extinction coefficient 239,000/M·cm, CF₂₈₀ 0.03 which is close to analytic and measured values of the compound 2-2 and the conventional dye. From the results of the experiments, it is analyzed that the labeling ratios by reaction ratio of two dyes are similar to each other.

Especially from FIG. 15, the compound 2-2 looks continuously linear until the reaction ratio of 33 Fold. The compound 2-2 labels the same protein better than the conventional dye.

b. Comparison of Fluorescent Intensity According to Chemical Labeling

The fluorescent intensity by the reaction ratio in the comparison experiment 4-(3)-a and the fluorescent intensities of the two dyes by the labeling ratio were compared.

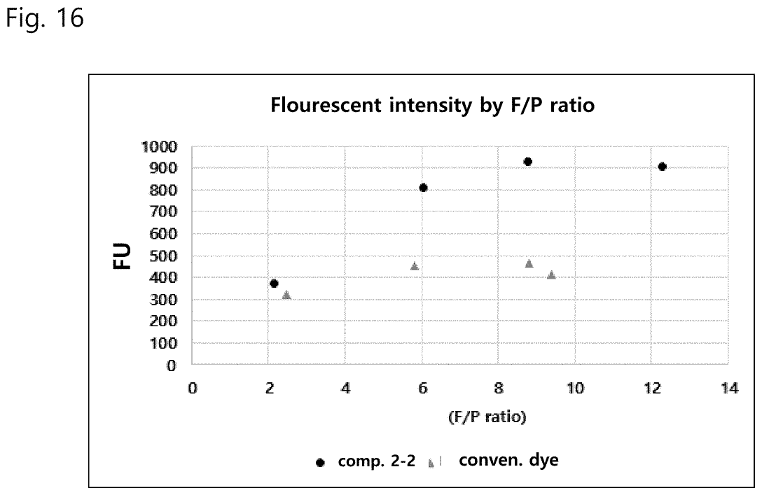

FIG. 16 shows the results, and the fluorescent intensities were measured by the PerkinElmer device.

It is measured that the fluorescent intensity of the compound 2-2 is higher than that of the conventional dye in all of the labeling ratio (reaction ratio).

In detail, when we compare the intensity of pseudo-labeling ratio, namely the ratio of about 5, 15, 25 Fold, between the objects the fluorescent intensity of the compound 2-2 to the antibody is more excellent than that of the conventional dye to the antibody.

In addition, in pseudo weight (mg) labeling the fluorescent intensity of the compound 2-2 is also stronger than that of the conventional dye. From this result, it is clearly verified that the affinity of the compound 22 to the target is the same or higher than that of the conventional dye (Flamma® 647 NHS ester).

### Comparative Example 5. Estimation of the Optical Characteristics of Compound 1-12

(1) Comparison of Absorption Characteristics

The absorption characteristics of the compound 1-12 in accordance with the present invention and a conventional dye (Invitrogen, Alexa Flour™ 750 NHS ester) were compared.

Each stock solution was prepared by adding the two dyes in DMF. The concentrations of the stock solutions were the same as 10 mg/ml.

First, to compare the absorption characteristics at the same molar concentration, the stock solutions were diluted by pH 7.4 1×PBS until the concentrations reach 5 uM and then the absorbances were analyzed by Agilent, Cary 3500 UV-Vis spectrophotometer.

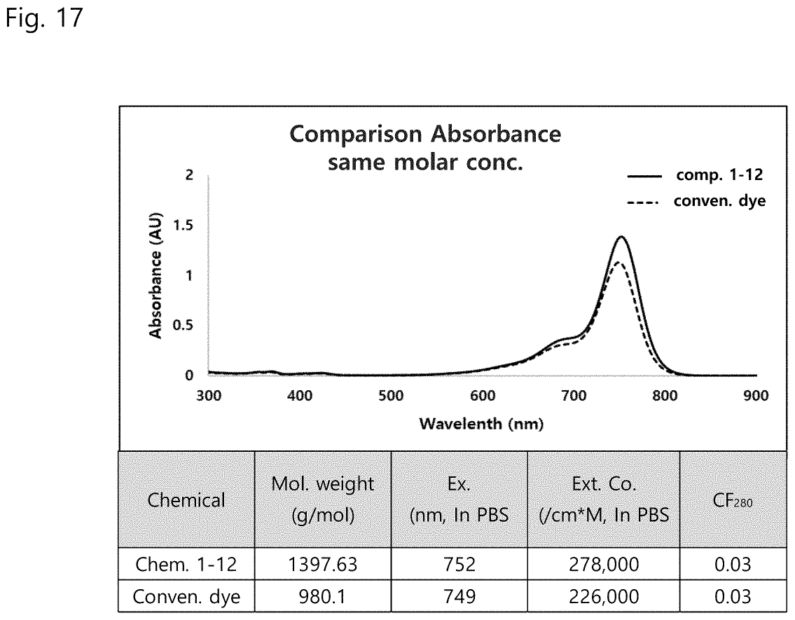

FIG. 17 shows the absorption characteristics of the two dyes. From FIG. 17, it is verified that the absorption intensity and the molar absorbance index of the compound 1-12 are higher than those of the conventional dye.

(2) The Comparison of Fluorescent Characteristics and Intensity

The fluorescent characteristics and intensity of the compound 1-12 and the conventional dye were compared.

Each stock solution was prepared by adding the two dyes in DMF. The concentrations of the stock solutions were the same as 10 mg/ml.

First, to compare the fluorescent intensity at the same molar concentration, the stock solutions were diluted by 1×PBS until the concentrations reach 0.1 uM and then the fluorescence were measured at the extinction wavelength of 750 nm.

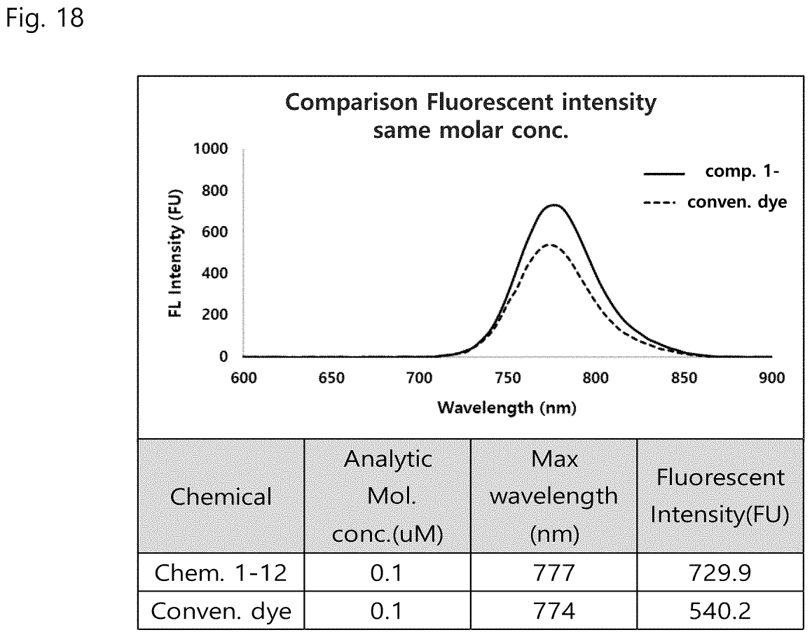

The fluorescence was measured by LS 55 Fluorescence spectrometer (PerkinElmer), and the results were shown in FIG. 18.

It shows that the fluorescent intensity of the compound 1-12 is higher than that of the conventional dye at the same molar concentration.

The maximum fluorescent wavelengths of the compound 1-12 and the conventional dye were verified as 777 nm, 774 nm respectively.

(3) Comparison of Performance after Protein Labeling

a. Comparison of the Ratio of Protein Labeling of Each Compound According to the Reaction Ratio

After antibody-labeling (Invitrogen, Goat anti Rabbit IgG H+L Secondary Ab, 150 kDa) of the compound 1-12 in accordance with the present invention and a conventional dye (Flamma® 647 NHS ester), the labeling ratio (F/P molar ratio) were measured and compared.

Before the labeling, the compound 1-12 and the conventional dye were dissolved in DMF, and the stock solution of 10 mg/ml was prepared. Thereafter the dyes were reacted with 0.1 mg of the antibody by each reaction ratio (2, 5, 15, 25, 33 Fold). The reaction buffer was prepared with the final pH of 8.3˜8.5, and the final concentration of the antibody was 2 mg/ml. The reaction was executed in 1 hour in dark environment at room temperature through stirring, and the reaction particles were separated and obtained through the column filled with Sephadex G-25 resin(Cytiva). The resin was used in a buffer equilibrium with 1×PBS.

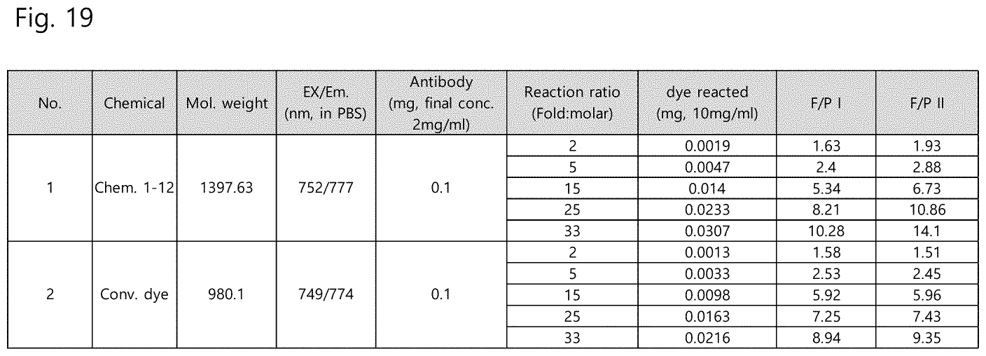

The fluorescent intensity of each reaction particle was measured at the wavelength of 280, 750 nm respectively (Agilent, Cary 3500 UV-Vis spectrophotometer), and the labeling ratio was calculated through a universal formula. The results were shown in FIG. 19.

The F/P ratios of the dyes were calculated by applying F/P I ratio, which is calculated value of each dye through analysis of molar absorption index and correction factor, and F/P II ratio, which is the ratio of the conventional dye described in the website (Extinction coefficient: 240,000, Correction factor: 0.04).

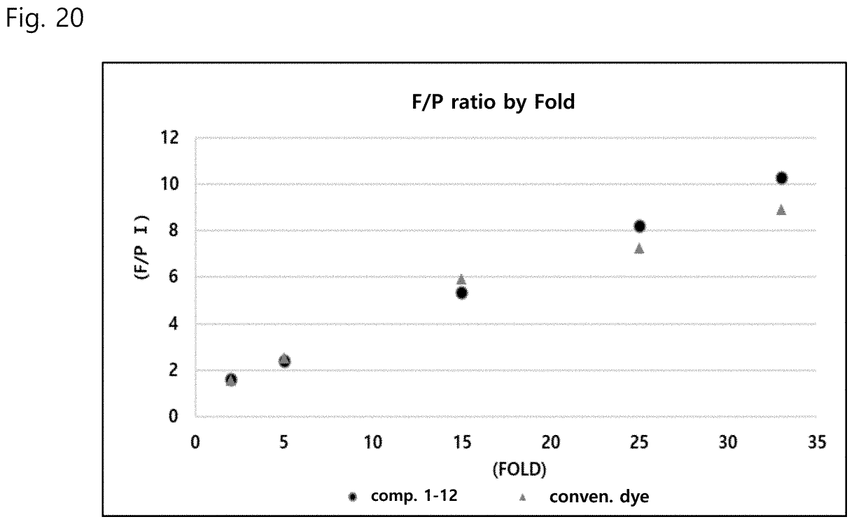

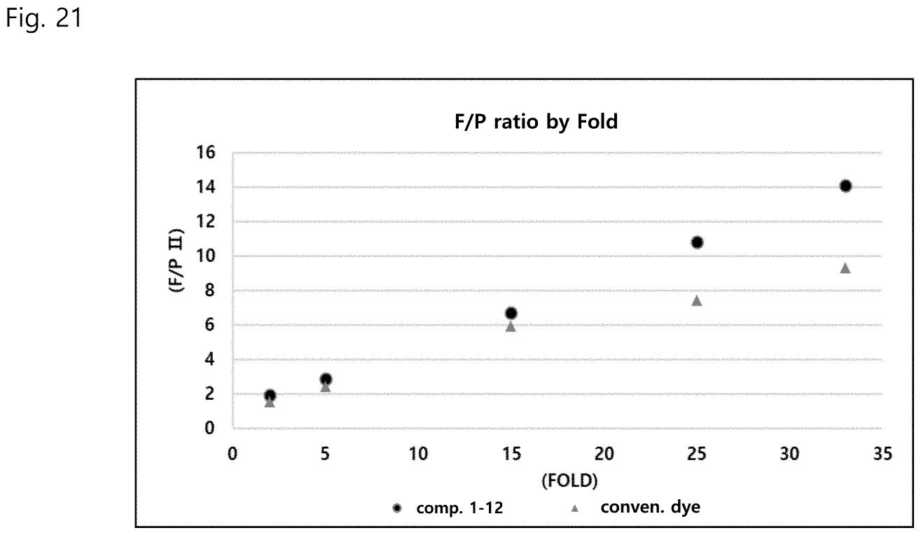

From the graph of labeling by F/P ratio (F/P I and II), the compound 1-12 looks more linear and higher slope than the conventional dye. The results are shown in FIG. 20 and FIG. 21.

b. Comparison of the Fluorescent Intensities Between Chemical and Antibody Conjugates

By using the fluorescent intensity by the reaction ratio in the comparison experiment 5-(3)-a, the fluorescent intensities of the two dyes by the labeling ratio were compared.

The conjugates were used without dilution, the intensity was measured at Excitation 750 nm by PerkinElmer LS 55 Fluorescence spectrometer.

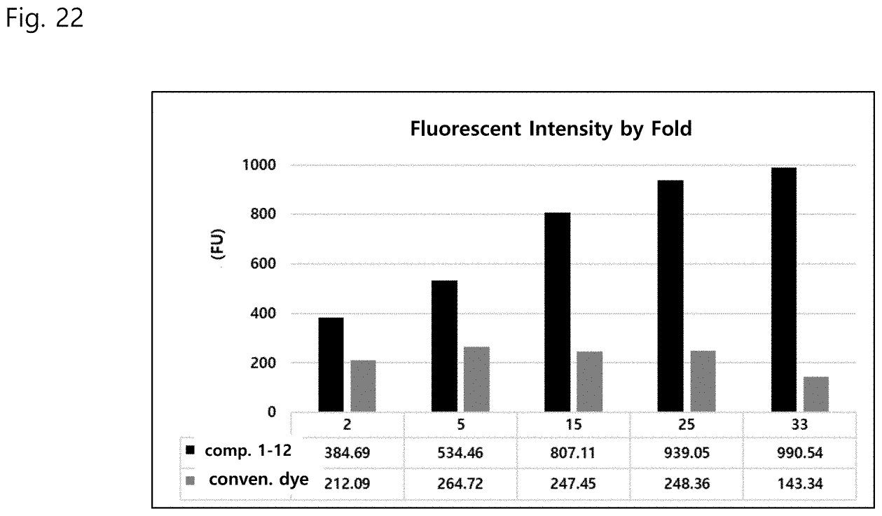

The results were shown in FIG. 22. FIG. 22 shows that the fluorescent intensity of the compound 1-12 is much higher than that of the conventional dye in all of the labeling ratio (reaction ratio).

Especially although the compound 1-12 was reacted in 2 Fold, the intensity of the compound 1-12 shows almost the same intensity as the highest intensity of the conventional dye. In addition, in the case of compound 1-12, the fluorescent intensity grows continuously stronger according to the increase of the reaction ratio which is different from the case of the conventional dye.

c. Comparison of Fluorescent Intensity According to Chemical Labeling

The fluorescent intensities of the two dyes by the labeling ratio were compared by measuring the fluorescent intensity by the reaction ratio in the said comparison experiment 5-(3)-a.

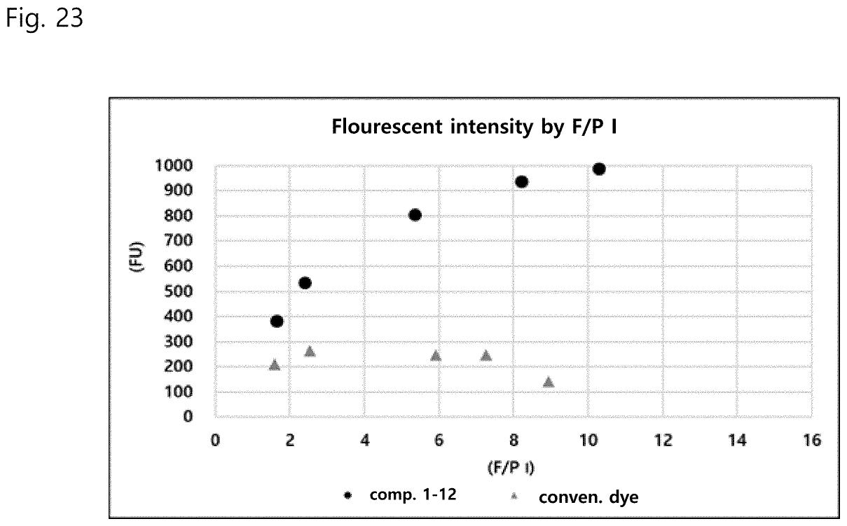

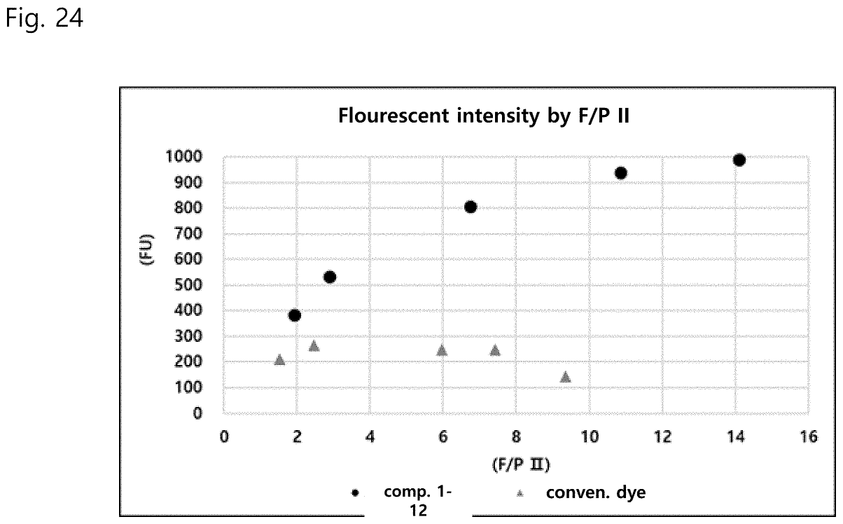

FIG. 23 and FIG. 24 show the results, and the fluorescent intensities were measured by the said PerkinElmer device.

It is measured that the fluorescent intensity of the compound 1-12 antibody conjugates is higher than that of the conventional dye in all of the labeling ratio (reaction ratio).

From the results, even at the lowest labeling ratio (about 3 Fold), the intensity of the compound 1-12 and antibody conjugate is higher than that of the conventional dye in all labeling ratios.

From the results, we can infer that although the less amount of compound 1-12 is used the higher fluorescent intensity can be shown than when a more amount of the conventional dye is used.

As described above, the fluorescent compound introducing a linker having a triazine structure substituted with a hydroxyl group to a cyanine-based fluorescent compound provided in exemplary embodiments of the present invention has high fluorescence efficiency such as fluorescent intensity and the like as compared with the conventional fluorescent compound at the same concentration to accurately detect a target material even in a small amount of biomaterial.

The present invention is not limited by the above-described embodiments, and various modifications and changes can be made by those skilled in the art and may be used in various biological and chemical fields, and are included in the spirit and scope of the present invention as defined in the appended claims.

1. A fluorescent compound for labeling a biomaterial having the following Chemical Formula 1: 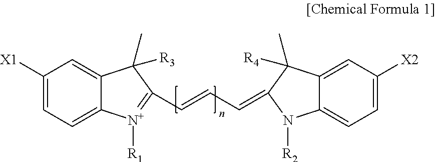
In Chemical Formula 1 above,
X1 and X2 are the same as or different from each other, and each independently selected from H, —SO₃⁻ and SO₃H,
R₁ and R₂ are the same as each other or each independently selected from C₁₋₇ alkyl, C₈₋₁₈ alkyl, —(CH₂)ₘSO₃⁻, —(CH₂)ₘSO₃H, and 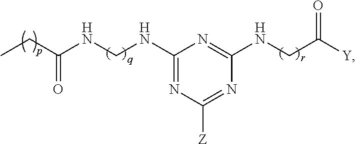
R₃ and R₄ are the same as or different from each other and each independently selected from C₁₋₇ alkyl, —(CH₂)ₘCOOZ and 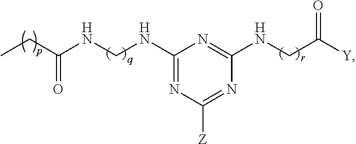
R₃ and R₄ are simultaneously not any one selected from —(CH₂)ₘCOOZ and 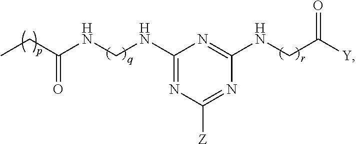
wherein,
n is an integer of 0 to 6,
m is an integer of 1 to 7,
p is an integer of 1 to 10,
q is an integer of 0 to 10,
r is an integer of 1 to 10, and
Z is OH or NH(CH₂)ₛSO₃H,
s is an integer of 1 to 7,
Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀₋₁₀ alkylamyl group, a vinylsulfonyl group, a vinylsulfonyl C₀₋₆ alkylaminyl group and an amino C₀₋₆ alkyl.

2. The fluorescent compound of claim 1, wherein the compound of Chemical Formula 1 above is any one selected from compounds represented by the following Chemical Formulas.

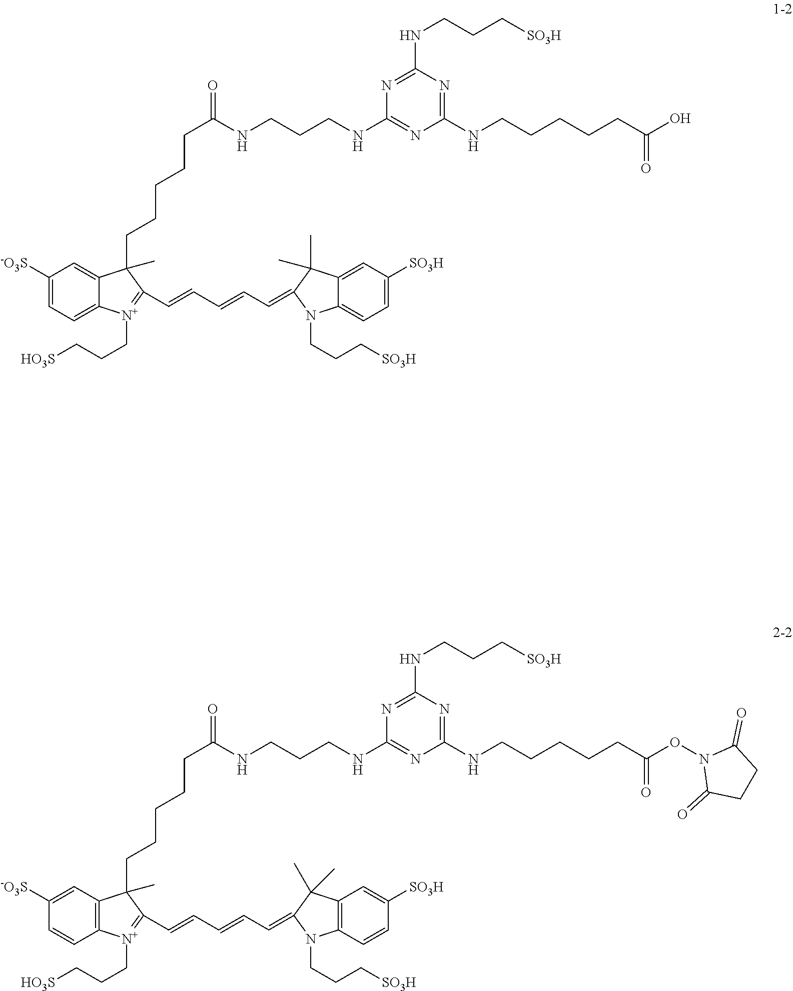

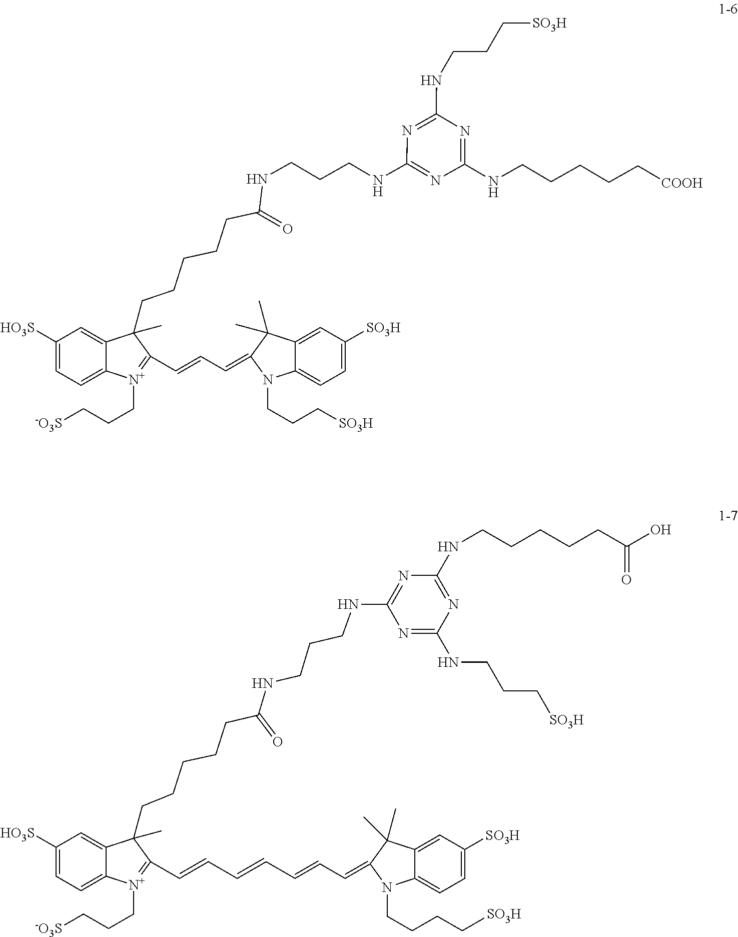

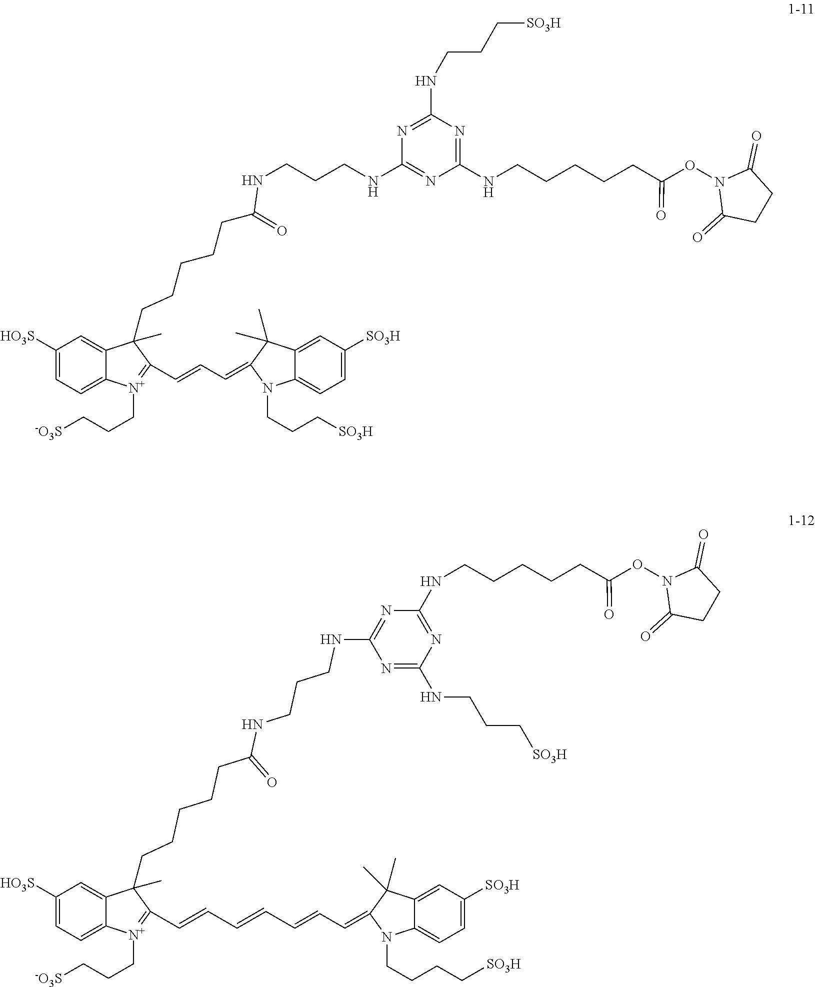

3. The fluorescent compound of claim 1, wherein the compound of Chemical Formula 1 above is any one selected from compounds represented by the following Chemical Formulas.

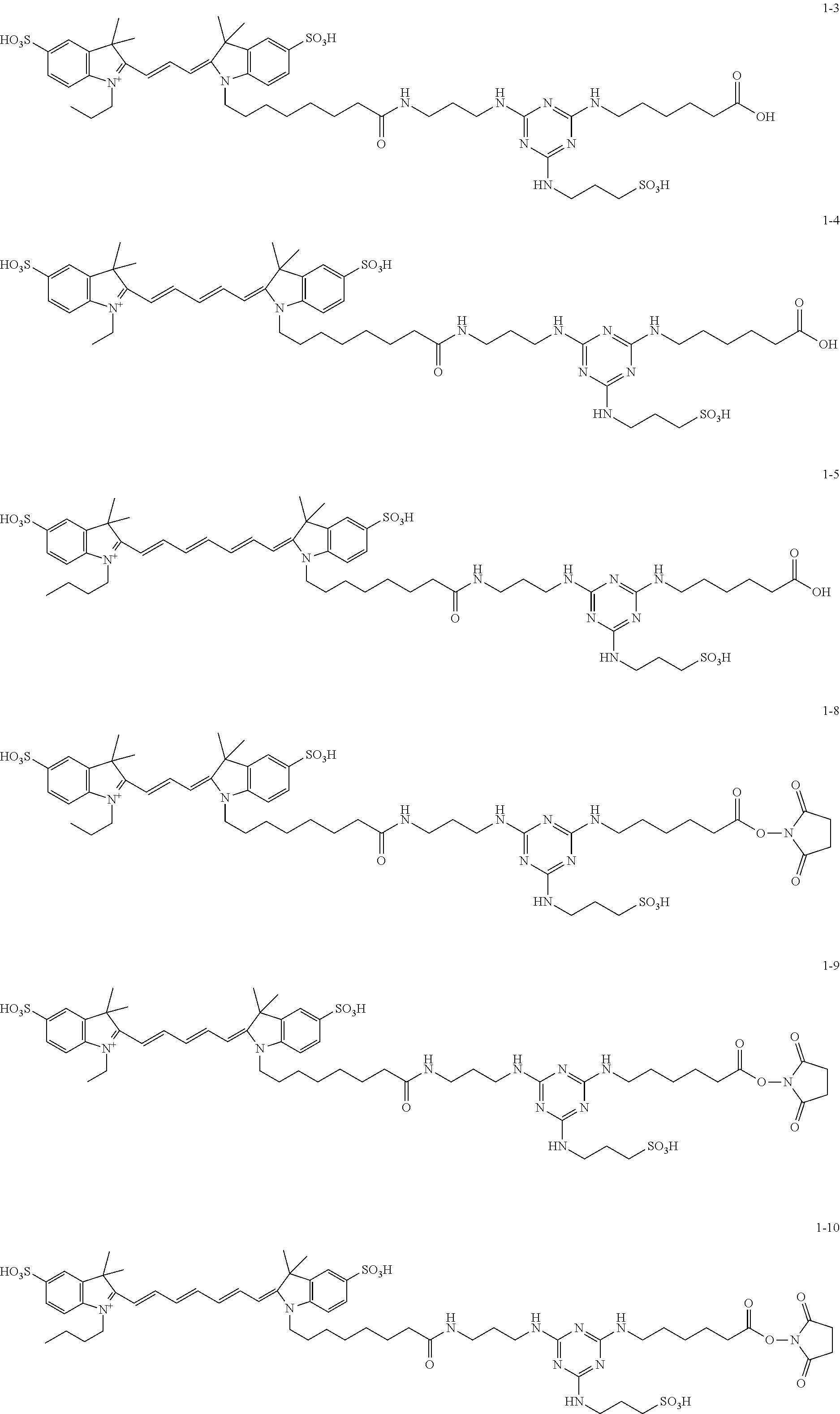

4. The fluorescent compound of claim 1, wherein the biomaterial is any one selected from the group consisting of proteins, peptides, carbohydrates, sugars, fats, antibodies, proteoglycan, glycoprotein, and siRNA.

5. A fluorescent diagnostic composition for detecting a biomaterial comprising a fluorescent compound represented by the following Chemical Formula 1: 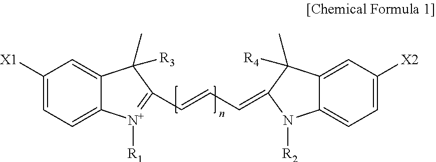
In Chemical Formula 1 above,
X1 and X2 are the same as or different from each other, and each independently selected from H, —SO₃⁻ and SO₃H,
R₁ and R₂ are the same as each other or each independently selected from C₁₋₇ alkyl, C₈₋₁₈ alkyl, —(CH₂)ₘSO₃⁻, —(CH₂)ₘSO₃H, and 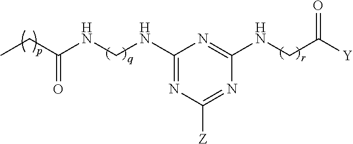
R₃ and R₄ are the same as or different from each other and each independently selected from C₁₋₇ alkyl, —(CH₂)ₘCOOZ and 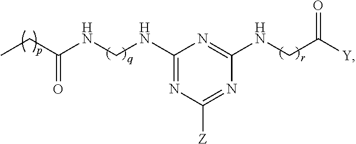
R₃ and R₄ are simultaneously not any one selected from —(CH₂)ₘCOOZ and 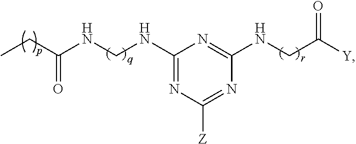
wherein,
n is an integer of 0 to 6,
m is an integer of 1 to 7,
p is an integer of 1 to 10,
q is an integer of 0 to 10,
r is an integer of 1 to 10, and
Z is OH or NH(CH₂)ₛSO₃H,
s is an integer of 1 to 7,
Y is selected from H, an N-succinimidol group, a hydrazinyl group, an N-hydroxysuccinimidyl group, an N-hydroxysuccinimidyl oxy group, a sulfosuccinimidyl oxy, a 4-sulfo-2,3,4,5-tetrafluoro phenyl group, a maleicimide C₀₋₁₀ alkylamyl group, a vinylsulfonyl group, a vinylsulfonyl C₀₋₆ alkylaminyl group and an amino C₀₋₆ alkyl.

6. The fluorescent diagnostic composition of claim 5, wherein the compound of Chemical Formula 1 above is any one selected from compounds represented by the following Chemical Formulas.

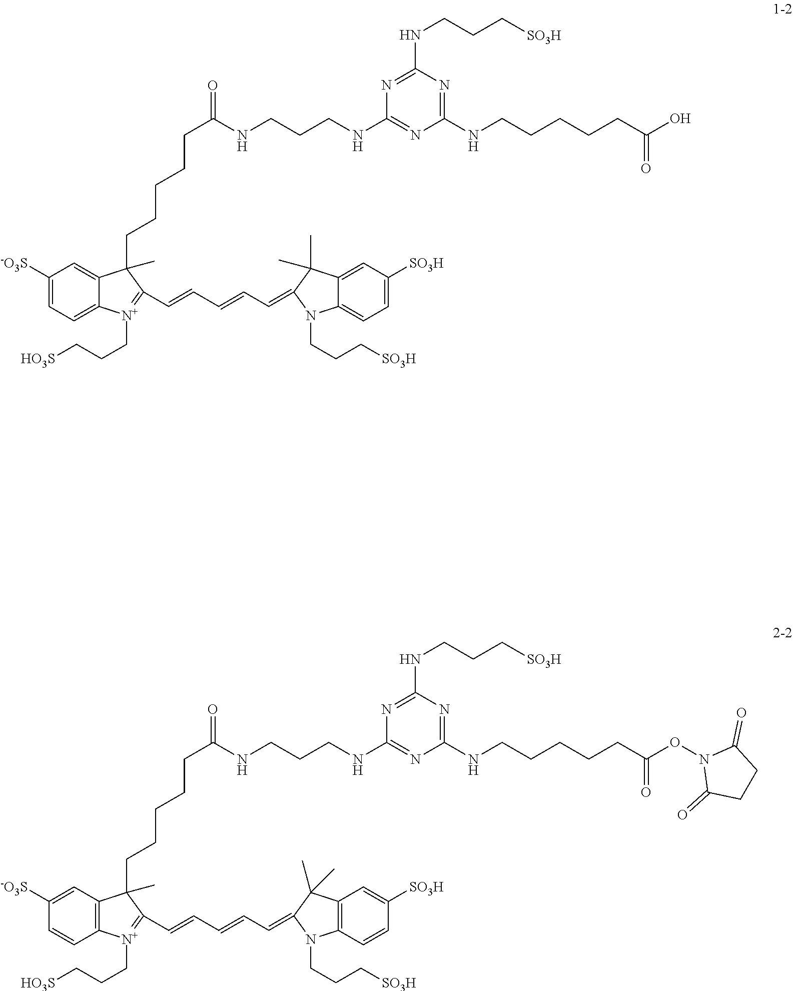

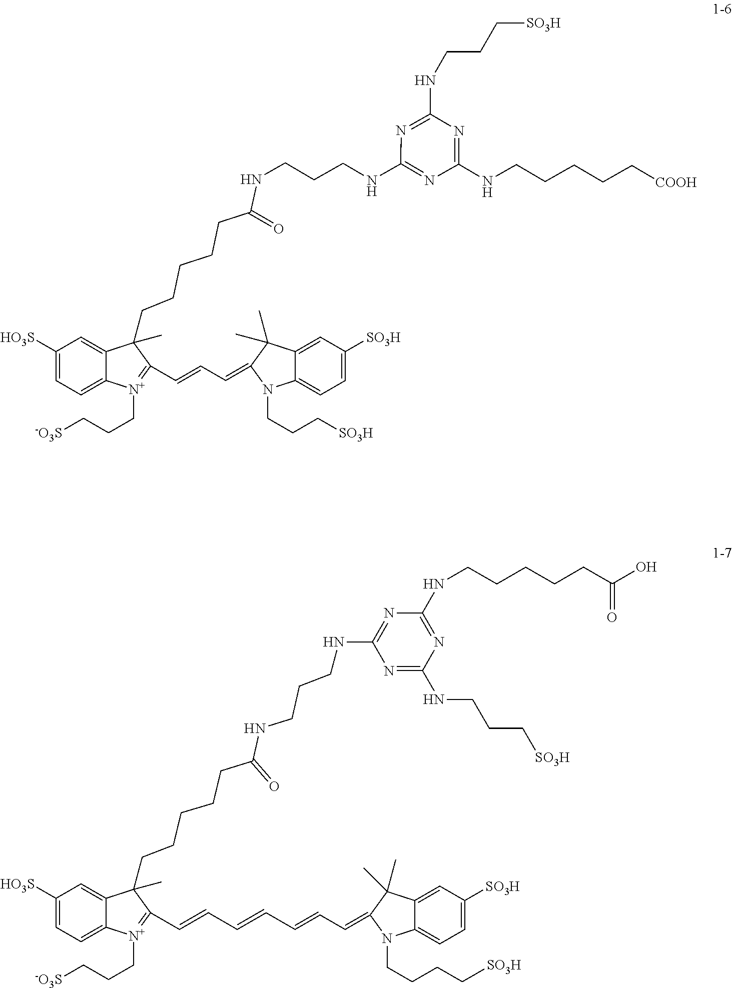

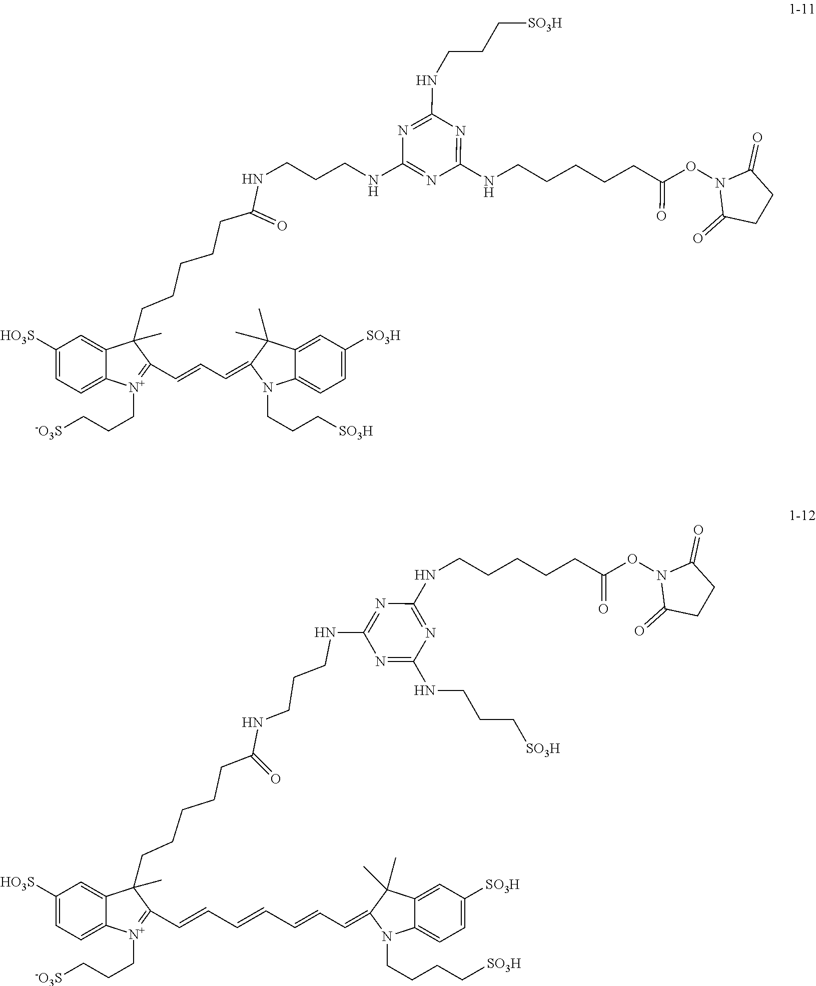

7. The fluorescent diagnostic composition of claim 5, wherein the compound of Chemical Formula 1 above is any one selected from compounds represented by the following Chemical Formulas.

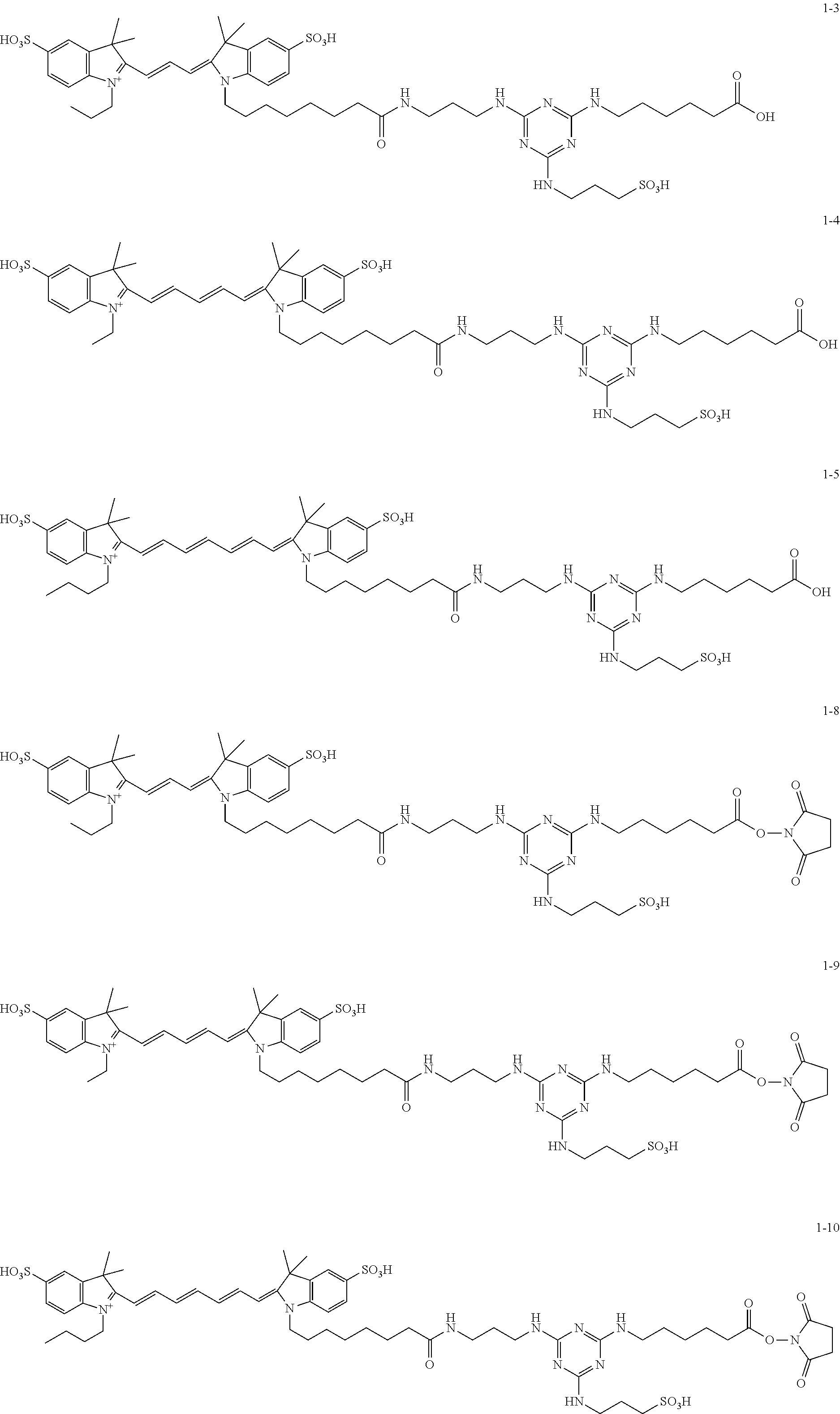

8. The fluorescent diagnostic composition of claim 5, wherein the biomaterial is any one selected from the group consisting of proteins, peptides, carbohydrates, sugars, fats, antibodies, proteoglycan, glycoprotein, and siRNA.
